SEQUENCE LISTING (20 sequences)

SEQ ID NO: 1 - DNA, 17 bp, Artificial Sequence
  primer 1
ccttgcacatgccggag

SEQ ID NO: 2 - DNA, 17 bp, Artificial Sequence
  primer 2
acagagcctcgcctttg

SEQ ID NO: 3 - DNA, 21 bp, Artificial Sequence
  probe
tcatccatggtgagctggcgg

SEQ ID NO: 4 - DNA, 23 bp, Artificial Sequence
  primer 1
ccattctgatttgctgccttatc

SEQ ID NO: 5 - DNA, 23 bp, Artificial Sequence
  primer 2
tactaatgctgatgcaggtacag

SEQ ID NO: 6 - DNA, 27 bp, Artificial Sequence
  probe
agtggcattcaaggagtacctctctct

SEQ ID NO: 7 - DNA, 22 bp, Artificial Sequence
  primer 1
aatacaggagtgacttggaact

SEQ ID NO: 8 - DNA, 19 bp, Artificial Sequence
  primer 2
aacagcgaccctttctcac

SEQ ID NO: 9 - DNA, 25 bp, Artificial Sequence
  probe
tcttgctggttctgattggagtgca

SEQ ID NO: 10 - DNA, 25 bp, Artificial Sequence
  primer 1
agttaatgctacagattgttatggc

SEQ ID NO: 11 - DNA, 20 bp, Artificial Sequence
  primer 2
tccgtgcaatatccagtgag

SEQ ID NO: 12 - DNA, 24 bp, Artificial Sequence
  probe
cccgtgcagaccccacaataaaga

SEQ ID NO: 13 - DNA, 21 bp, Artificial Sequence
  primer 1
gatccagtgagagacacagaa

SEQ ID NO: 14 - DNA, 18 bp, Artificial Sequence
  primer 2
catgtccaggctgttccc

SEQ ID NO: 15 - DNA, 24 bp, Artificial Sequence
  probe
tgctagaagccagtgctcgtgaac

SEQ ID NO: 16 - DNA, 27 bp, Artificial Sequence
  primer 1
tcggtaactgacttgaatgtccaacgc

SEQ ID NO: 17 - DNA, 20 bp, Artificial Sequence
  primer 2
cgacagttcagccatcactt

SEQ ID NO: 18 - DNA, 20 bp, Artificial Sequence
  primer 1
aaactcaccaggatgctcac

SEQ ID NO: 19 - DNA, 22 bp, Artificial Sequence
  primer 2
ctccagaggtttgagttcttct

SEQ ID NO: 20 - DNA, 24 bp, Artificial Sequence
  probe
ttacatgcccaagaaggccacaga

(19) United States
(12) Patent Application Publication
(10) Pub. No.: US 2025/0369049 A1
(43) Pub. Date: Dec. 4, 2025

(54) METHODS FOR DETECTING AND STAGING CELLULAR VIRAL IMMUNE RESPONSES

(71) Applicant: Icahn School of Medicine at Mount Sinai, New York, NY (US)

(72) Inventors: Jordi Ochando, New York, NY (US); Ernesto Guccione, New York, NY (US); Denis Torre, New York, NY (US); Daniel Lozano-Ojalvo, New York, NY (US)

(73) Assignee: ICAHN SCHOOL OF MEDICINE AT MOUNT SINAI, New York, NY (US)

(21) Appl. No.: 18/560,052

(22) PCT Filed: May 10, 2022
(86) PCT No.: PCT/US2022/028608

§ 371 Date: Nov. 9, 2023

Related U.S. Application Data
(60) Provisional application No. 63/186,559, filed on May 10, 2021.

(51) Int. Cl. C12Q 1/6883

(52) U.S. Cl. CPC C12Q 1/6883; C12Q 2600/158

(57) ABSTRACT

The present disclosure relates to methods for detecting and staging cellular immune responses to viral infections, including to SARS-CoV-2 and to methods for treating viral infections.

### RELATED APPLICATION

This application is a § 371 national stage of PCT International Application No. PCT/US22/028608, filed May 10, 2022, claiming the benefit claims priority to U.S. Provisional Application No. 63/186,559, filed May 10, 2021, which is incorporated herein by reference in its entirety.

### SEQUENCE LISTING

The instant application contains a Sequence Listing which has been submitted via Patent Center and is hereby incorporated by reference in its entirety. Said .txt copy, created on Jun. 6, 2024 is named MS-0031-01-US-NP_ST25, and is 4,013 bytes in size.

### BACKGROUND

Embodiments of the present disclosure relate generally to methods of detecting cellular viral immune responses (including to acute respiratory syndrome coronavirus 2 (SARS-CoV-2)) and to treating, staging, and preventing such viral infections.

Infection with the severe acute respiratory syndrome coronavirus 2 (SARS-CoV-2), the etiological agent that causes the COVID-19 disease, is now a pandemic resulting in a global health crisis, with over 510 million global cases resulting in over 6.2 million deaths as of May 2022 [https://coronavirus.jhu.edu/map.html]. SARS-CoV-2 infection leads to a broad spectrum of clinical syndromes, ranging from asymptomatic to severe pneumonia and Acute Respiratory Distress Syndrome (ARDS) (Bhatraju, P. K. et al. (2020); Wu, C. et al. (2020)). Due to an unprecedented effort by the global scientific community, the deployment of mRNA and viral vector-based vaccines has begun to efficiently attenuate this worldwide crisis (Dagan, N. et al. (2021)). Nonetheless, vaccine effectiveness and duration of protective immunity will need to be systematically assessed and monitored at a global level.

Long-term protection from viral infections is mediated by both the humoral (antibodies) and cellular immune pathways (McMahan, K. et al. (2021)). While SARS-CoV-2-specific IgG and neutralizing antibody quantification are being used as clinical endpoints to determine immune protection (Wajnberg, A. et al. (2020)), a precise measurement of cellular responses underlying virus protection also represents an important parameter of immune defense, which is rarely performed due to the associated technical challenges.

Several groups have been quantifying SARS-CoV-2-specific T cells using synthetic peptides (15-mers long) to activate T cells in vitro following overnight incubation with whole blood. These peptides are either presented directly by HLA-class II or possibly processed by proteases present in the blood and presented by HLA-class I. Previous studies have demonstrated that these peptides activate SARS-CoV-2 specific CD4 and CD8 T cells (Borobia, A. (2021); Le Bert, N. et al. (2020); Hillus, D. et al. (2021); Lozano-Ojalvo, D. et al. (2021).

Recently, the inventors demonstrated that the same peptides used in the present disclosure activate SARS-CoV-2 T cells in Peripheral Blood Mononuclear Cells (PBMCs) and in whole blood (Le Bert, N. et al. (2021). The inventors have also demonstrated that the quantity of cytokines (IL-2 and IFN-γ) measured by ELISA in the whole blood after overnight stimulation correlates with the number of SARS-CoV-2 specific T cells quantified with ELISpot, which they further confirmed by intracellular cytokine staining (ICS). This data collectively confirms that the addition of peptides directly in whole blood allows precise quantification of SARS-CoV-2 specific T cells (Tan, A. T. et al. (2021); Le Bert, N. et al. (2021); Petrone, L. et al. (2021); Murugesan, K. et al. (2021)).

Despite the recognized need to quantify the levels of cellular immunity, the complexity and lack of scalability of these traditional methods (i.e. ELISpot and flow cytometry), has so far prevented large scale studies of the cellular immune response to COVID-19 recovered and vaccinated individuals. To illustrate this inherent lack of scalability, most studies utilizing ELISpot or flow cytometry assess between 10 and 40 subjects, with larger clinical trials assessing around 200 (Zhu, F. C. et al. (2020); Folegatti, P. M. et al. (2020); Zhu, F. C. et al. (2020); Kroemer, M. et al. (2021); Ramasamy, M. N. et al. (2021); Prendecki, M. et al. (2021)). Furthermore, the process of freezing/thawing PBMCs, often utilized for testing T cell response, can introduce high variability in the results (Tan, A. T. et al. (2021); Ford, T. et al. (2017)), issues that can be bypassed by using whole blood.

Thus, there is a need for fast, high-throughput methods of detecting, staging, and treating viral infections (including from SARS-CoV-2) that are effective, accurate, efficient, and scalable.

### SUMMARY

The present disclosure solves these problems by providing for the first-time a rapid, scalable assay that can be employed to readily detect T-cell activation by viruses, including SARS-CoV-2, and that can be used as a marker for diagnosing, staging, and treating such viral infections. It can also serve as a method for assessing the immune response to SARS-CoV-2 and related vaccines and for determining when to vaccinate or revaccinate for SARS-CoV-2.

Embodiments of the present disclosure relate generally to methods of preventing, staging, diagnosing, and treating SARS-CoV-2 infections in patients.

### Definitions

Unless defined otherwise, all technical and scientific terms used herein have the meaning commonly understood by a person skilled in the art to which this disclosure belongs. The meaning and scope of the terms should be clear; however, in the event of any latent ambiguity, definitions provided herein take precedent over any dictionary or extrinsic definition.

As used herein, the following terms have the meanings ascribed to them below, unless specified otherwise. The terminology used herein is for the purpose of describing particular embodiments only and is not intended to be limiting of the disclosure. As used herein, the singular forms “a”, “an,” and “the” are intended to include the plural forms as well, unless the context clearly indicates otherwise.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “pool of polypeptides” refers to polypeptides that are encoded by sequences unique to a particular virus. Such polypeptides may include the complete polypeptide sequences or fragments of polypeptides that are encoded by viral polynucleotides.

As used herein, the terms “at least one viral spike protein,” “at least one nucleoprotein,” and “at least one non-structural protein” refer to the complete proteins encoded by the genome of a particular virus or to fragments of the encoded proteins. The terms “at least one SARS-CoV-2 viral spike protein”, “at least one SARS-CoV-2 nucleoprotein,” and “at least one SARS-CoV-2 non-structural protein,” refer to the complete proteins encoded by the genome of a SARS-CoV-2 virus or mutant virus or to fragments of the encoded proteins.

As used herein, the term “housekeeping gene” or “house-keeping gene” refers to constitutive genes that are required for the maintenance of basal cellular functions essential to the existence of a cell and that are typically used as controls in qPCR reactions. A non-exhaustive list of such genes includes the following: ACTIN, RRN18S, GAPDH, PGK1, B2M, and other such genes that a person of skill in the field would understand are included in this term.

As used herein, the term “buffer” refers to a solution that contains water and optionally other ingredients, including, for example, detergents, ions, nucleotides, proteins, and other ingredients.

As used herein, the terms “comprising” and “including” are used in their open, non-limiting sense.

The term “subject,” as used herein, refers to any animal. In some instances, the subject is a mammal. In some instances, the term “subject,” as used herein, refers to a human (e.g., a man, a woman, or a child).

The terms “administer,” “administering,” or “administration,” as used herein, refer to implanting, ingesting, injecting, inhaling, or otherwise absorbing a compound or composition, regardless of form. For example, the methods disclosed herein include administration of an effective amount of a compound or composition to achieve the desired or stated effect.

The terms “treat”, “treating,” or “treatment,” as used herein, refer to partially or completely alleviating, inhibiting, ameliorating, or relieving the disease or condition from which the subject is suffering. This means any way that one or more of the symptoms of a disease or disorder are ameliorated or otherwise beneficially altered. As used herein, amelioration of the symptoms of a particular disorder refers to any lessening, whether permanent or temporary, lasting, or transient that can be attributed to or associated with treatment by the compositions and methods of the present invention. In some aspects, treatment can promote or result in, for example, reductions in one or more symptoms associated with a SARS-CoV-2 infection in a subject relative to the subject's symptoms prior to treatment.

The terms “prevent,” “preventing,” and “prevention,” as used herein, shall refer to a decrease in the occurrence of a disease or decrease in the risk of acquiring a disease or its associated symptoms in a subject. The prevention may be complete, e.g., the total absence of disease or pathological cells in a subject. The prevention may also be partial, such that the occurrence of the disease or pathological cells in a subject is less than, occurs later than, or develops more slowly than that which would have occurred without the present invention.

As used herein, the term “preventing a disease” in a subject means for example, to stop the development of one or more symptoms of a disease in a subject before they occur or are detectable, e.g., by the patient or the patient's doctor. Preferably, the disease does not develop at all, i.e., no symptoms of the disease are detectable. However, it can also mean delaying or slowing of the development of one or more symptoms of the disease. Alternatively, or in addition, it can mean decreasing the severity of one or more subsequently developed symptoms.

Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician. Following administration, the subject can be evaluated to detect, assess, or determine their level of disease.

In one aspect, the present disclosure provides a kit for detecting a cellular immune response to a virus in at least one subject, wherein the kit comprises: reverse transcriptase; polymerase; dNTPs; primers/probes targeting CXCL10 and primers/probes targeting a house-keeping gene; reaction buffer; PCR enhancer cocktail; and optionally, a buffer comprising a detergent. In an embodiment, the kit further comprises a pool of polypeptides consisting essentially of polypeptides derived from sequences unique to SARS-CoV-2 or a variant thereof, wherein said pool optionally includes polypeptides derived from at least one spike protein, at least one nucleoprotein, at least one non-structural protein, or combinations thereof. In an embodiment, said house-keeping gene is ACTIN. In an embodiment, said detergent is selected from the group consisting of: Tween; Triton; and n-dodecyl-β-D-maltoside (DDM). In an embodiment, said kit includes a detergent buffer for pretreating the tissue sample prior to performing PCR. In an embodiment, said detergent buffer comprises detergent at a concentration of about 0.0 g/mL to about 0.55 g/mL.

In one aspect, the present disclosure provides a method for detecting in a subject the presence of T-cells specific for a particular virus, wherein: a tissue sample from said subject has been stimulated for a period of time via incubation with a pool of polypeptides unique to said virus; at least one quantitative PCR (qPCR) assay has been performed on said stimulated tissue sample; results have been obtained that quantify the relative concentration of CXCL10 mRNAs in the sample; said results have been compared with a reference standard; and the results of the comparison indicate whether said subject has T-cells specific for said virus. In an embodiment, said virus is SARS-CoV-2 and said pool of polypeptides contains at least one viral spike protein, at least one viral nucleoprotein, at least one non-structural protein, or combinations thereof. In an embodiment, said tissue sample is blood and said blood has been diluted prior to performing the at least one qPCR assay. In an embodiment, said tissue sample is mixed with a buffer prior to performing the at least one qPCR assay to generate a mixture comprising a final detergent concentration of about 0.0 g/mL to about 0.1 g/mL. In an embodiment, said buffer comprises the detergent Triton or the detergent Tween and said mixture comprises a final detergent concentration of about 0.0 g/mL to about 0.1 g/mL. In an embodiment, said buffer comprises Tween-20 and said mixture comprises a final detergent concentration of about 0.0 g/mL to about 0.1 g/mL. In an embodiment, the blood is diluted prior to performing qPCR between a ratio of about 1:1 to about 1:5 to make the sample mixture.

In one aspect, the present disclosure provides a method where: a tissue sample has been stimulated via incubation with a pool of polypeptides derived from SARS-CoV-2 or a mutated variant thereof for a period of time; at least one quantitative PCR (qPCR) assay has been performed on said stimulated tissue sample; the relative concentration of CXCL10 mRNAs in the sample has been obtained; and wherein, the results of said assay have been compared with a reference standard; and determining from said results whether the subject has SARS-CoV-2 specific T-cells. In an embodiment, said pool of SARS-CoV-2 viral peptides contains at least one viral spike protein, at least one viral nucleoprotein, at least one non-structural protein, or combinations thereof. In an embodiment, said tissue sample is blood and polynucleotides have been isolated from said sample prior to the performance of the at least one qPCR assay. In an embodiment, said tissue sample is blood and said blood has been diluted prior to the performance of the at least one qPCR assay.

In one aspect, the present disclosure provides method for detecting an immune response to SARS-CoV-2 in a subject, wherein: a tissue sample from the subject has been stimulated via incubation with a pool of polypeptides derived from SARS-CoV-2 or a mutated variant thereof for a period of time; at least one quantitative PCR (qPCR) assay has been performed on said stimulated tissue sample; results have been obtained that quantify the relative concentration of CXCL10 mRNAs in the sample; said results have been compared with a reference standard. In an embodiment, the results of the comparison indicate whether to vaccinate the subject for SARS-CoV-2. In an embodiment, said pool of SARS-CoV-2 derived polypeptides contains fragments or complete polypeptides from at least one spike protein, at least one nucleoprotein, at least one non-structural protein, or combinations thereof. In an embodiment, the method further comprises providing a SARS-CoV-2 vaccination to the subject. In an embodiment, said tissue sample is blood and said blood has been diluted prior to the performance of the at least one qPCR assay. In an embodiment, said tissue sample is diluted using a buffer, optionally containing detergent at a concentration of about 0.0 g/mL to about 0.1 g/mL.

In one aspect, the present disclosure provides a method where: a tissue sample has been stimulated via incubation with a pool of SARS-CoV-2 viral peptides for a period of time; at least one quantitative PCR (qPCR) assay has been performed on said stimulated tissue sample; the relative concentration of CXCL10 mRNAs in the sample has been obtained; and wherein, the results of said assay have been compared with a reference standard; and determining from said results whether to administer a SARS-CoV-2 vaccine. In an embodiment, said pool of SARS-CoV-2 viral peptides contains at least one viral spike protein, at least one viral nucleoprotein, at least one non-structural protein, or combinations thereof. In an embodiment, said tissue sample is blood and said blood has been diluted prior to the performance of the at least one qPCR assay.

In one aspect, the present disclosure provides a method for treating SARS-CoV-2 infection in a subject in need thereof, where: a tissue sample from said subject has been stimulated by incubation with a pool of polypeptides derived from SARS-CoV-2 or a mutated variant thereof for a period of time; at least one quantitative PCR (qPCR) assay has been performed on the stimulated tissue sample; results have been obtained that quantify the relative concentration of CXCL10 mRNAs in the sample; said results have been compared with a reference standard; said results of the comparison indicate whether to treat the subject for a SARS-CoV-2 infection; and providing or withholding treatment for SARS-CoV-2. In an embodiment, said pool of polypeptides contains fragments or complete polypeptides from at least one spike protein, at least one nucleoprotein, at least one non-structural protein, or combinations thereof. In an embodiment, a SARS-CoV-2 vaccination is provided to the subject. In an embodiment, said treatment includes administering a therapeutic selected from the group consisting of: antivirals, corticosteroids, monoclonal antibodies, NSAIDs, convalescent plasma, or antidepressants. In an embodiment, said tissue sample is blood and said blood has been diluted prior to the performance of the at least one qPCR assay.

In one aspect the present disclosure provides a method where: a tissue sample has been stimulated via incubation with a pool of SARS-CoV-2 viral peptides for a period of time; at least one quantitative PCR (qPCR) assay has been performed on said stimulated tissue sample; the relative concentration of CXCL10 mRNAs in the sample has been obtained; and wherein, the results of said assay have been compared with a predetermined reference standard; and determining from said results whether the subject shows T-cell activation by SARS-CoV-2. In an embodiment, said pool of SARS-CoV-2 viral peptides contains at least one viral spike protein or at least one viral nucleoprotein. In an embodiment, said tissue sample is blood and polynucleotides have been isolated from said sample prior to the performance of the at least one qPCR assay. In an embodiment, said tissue sample is blood and said blood has been diluted prior to the performance of the at least one qPCR assay. In an embodiment, said method further comprises determining whether to administer a SARS-CoV-2 vaccine.

In one aspect, the present disclosure provides a method for gauging the immune status of a subject with respect to a specific virus, wherein: a tissue sample from said subject has been stimulated via incubation with a pool of polypeptides derived from sequences specific to said virus or mutated variants of said virus for a period of time; at least one quantitative PCR (qPCR) assay has been performed on said stimulated tissue sample; results have been obtained that quantify the relative concentration of CXCL10 mRNAs in the sample; said results have been compared with a reference standard; said results of the comparison indicate whether to treat or vaccinate the subject for an infection from said virus; and providing or withholding treatment or vaccination for said viral infection. In an embodiment, said pool of polypeptides contains fragments or complete polypeptides from at least one spike protein, at least one nucleoprotein, at least one non-structural protein, or combinations thereof. In an embodiment, a vaccination for said virus is provided to the subject. In an embodiment, said treatment includes administering a therapeutic selected from the group consisting of: antivirals, corticosteroids, monoclonal antibodies, NSAIDs, convalescent plasma, or antidepressants. In an embodiment, said tissue sample is blood and said blood has been diluted prior to the performance of the at least one qPCR assay.

### BRIEF DESCRIPTION OF THE DRAWINGS

The accompanying figures, which are incorporated in and constitute a part of this specification, illustrate several aspects described below. The figures do not limit the scope of the invention.

FIGS. 1A-E. The figure shows that CXCL10 mRNA levels can be measured as a proxy for T cell activation. A. Schematic of workflow for the T cell activation assays. All assays begin with whole blood collection followed by overnight stimulation with DMSO or nucleocapsid (NP) or spike (SpG) peptide pools. Next, supernatants are collected for ELLA or Olink assays; RNA is extracted and used for probe-based qPCR (i.e. BioRad CFX96/384 or Hyris bCUBE 2.0) or NGS (Illumina); or whole blood ix diluted and used directly for qPCR (i.e. BioRad CFX96/384 or Hyris bCUBE 2.0). B. CXCL10 is upregulated in response to Spike peptide pool activation of whole blood. Differentially expressed genes stimulated in whole blood by the spike peptide pool versus DMSO, grouped by the subject's COVID19 or vaccination status, displayed as red (upregulated) or blue (downregulated) dots. Significantly differentially expressed genes were defined as having a p-value <0.05 and log 2FC>1. P-values were calculated using DESeq2 and adjusted using the Benjamini-Hochberg method. C. CXCL10 and IFNG mRNA induction correlate. Differentially expressed genes' (log 2FC for the spike peptide pool versus DMSO: Y-axis) correlation with IFNG expression (X-axis), grouped by COVID19 or vaccination status of the cohort. D. Venn diagram displaying overlap in significantly upregulated genes in convalescent and vaccinated subjects for spike peptide stimulated samples compared to DMSO control samples. Significantly differentially expressed genes were defined as having a p-value <0.05 and log 2FC>1. E. Gene set enrichment analysis (GSEA) plot—displaying significantly positive enrichment for IFN-γ response genes among the upregulated genes in spike peptide simulate samples compared to DMSO control samples in COVID-19 convalescent and vaccinated cohorts. GSEA was performed using fgsea (v1.16.0).

FIGS. 2A-E. The figure shows CXCL10 is upregulated by monocytes in response to IFN-γ released by antigen-specific T cells. A. Schematic of proposed mechanism of CXCL10 transcript upregulation. Upon spike stimulation of whole blood, antigen presenting cells present spike peptides to antigen-specific T cells that subsequently release IFN-γ. IFN-γ release can be quantified by ELLA, ELISpot, or flow cytometry. Next, IFN-γ stimulates monocytes, which, in turn, upregulate CXCL10 mRNA, which can be detected by the qTACT/dqTACT assays. B. Only monocytes upregulate CXCL10 in response to IFN-γ and TNF-α. Whole blood was stimulated with DMSO (Neg) or IFN-γ+TNF-α in the presence of brefeldin/Monensin (BFA/Mon). CXCL10/IP-10 positive T cells (T), B cells (B), natural killer cells (NK), natural killer T cells (NK-T), monocytes (Mono), and neutrophils (Neutro) were quantified using flow cytometry. P-values were calculated using a two-sided Wilcoxon matched-pairs signed rank test. N=4 biologically independent samples. C. Both monocytes and neutrophils release CXCL10/IP-10 upon SpG stimulation. Whole blood was stimulated with DMSO (Neg) or a spike peptide pool (SpG) in the absence (left panel) or presence (right panel) of BFA/Mon. CXCL10 positive T, B, NK, NK-T, Mono, and Neutro were quantified using flow cytometry. P-values were calculated using a two-sided Wilcoxon matched-pairs signed rank test. N=4 biologically independent samples. D. Monocytes, and not neutrophils, upregulate CXCL10/IP-10 in response to SpG. Whole blood was simulated with DMSO (Neg) or a spike peptide pool (SpG) overnight with BFA/Mon added for the last 4 hours. CXCL10 positive Monocytes and Neutrophils were quantified using flow cytometry. P-values were calculated using a two-sided Wilcoxon matched-pairs signed rank test (p=0.015625). N=7 biologically independent samples. E. CXCL10 mRNA is upregulated in monocytes upon stimulation with spike peptides. Monocytes and neutrophils were sorted from whole blood after overnight stimulation with spike peptides or DMSO control. RNA was extracted from the cells and the relative CXCL10 mRNA expression was determined using the qTACT assay. P-values were calculated using a two-sided Wilcoxon matched-pairs signed rank test (p=0.03125). N=7 biologically independent samples. For B-E, the box bounds represent the first quartile (bottom), median (center), and the third quartile (top). The whiskers represent the range of samples up to 1.5 times the interquartile range. Beyond this point, samples are shown as outliers.

FIGS. 3A-D. The figure shows that TACT assays are concordant with gold standard ELLA and ELISpot assays. A-D. Concordance between assays to quantify cellular immunity. The quantification shown is with the DMSO control subtracted from the spike peptide stimulated sample. Each dot represents a unique subject color coded based on their COVID-19 and vaccination statuses (see legend). The dashed line represents thresholds for each assay. A. Concordance between ELLA and qTACT assays. For 117 subjects, IFN-γ protein secretion was quantified by ELLA (y-axis) and CXCL10 mRNA by qTACT (x-axis). B. Concordance between ELLA and dqTACT assays. For 133 subjects, IFN-γ protein secretion was quantified by ELLA (y-axis) and CXCL10 mRNA by dqTACT (x-axis). C. Concordance between ELISpot and qTACT assays. For 50 subjects, IFN-γ producing cells were quantified by ELISpot (y-axis) and CXCL10 mRNA by qTACT (x-axis). D. Concordance between ELISpot and dqTACT assays. For 46 subjects, IFN-γ producing cells were quantified by ELISpot (y-axis) and CXCL10 mRNA by dqTACT (x-axis).

FIGS. 4A-B. The figure provides analytical validation and comparison of available T cell assays. A. Comparison of the various assays used to determine T-cell response to spike peptides. Total P/N=total positives/negatives (i.e. above or below threshold, respectively). TP/TN=true positives/negatives (i.e. correctly above or below threshold, respectively, according to the subject's COVID-19/vaccination status). FP/FN=false positives/negatives. Sensitivity=true positives/(true positives+false negatives). Specificity=true negatives/(true negatives+false positives). Diagnostic accuracy=(true positives+true negatives)/all samples). B. ROC curves and associated 95% confidence intervals for each assay.

FIGS. 5A-D. The figure shows the use of qTACT to monitor cellular immunity in vaccinated subjects. For A-D, the box bounds represent the first quartile (bottom), median (center), and the third quartile (top). The whiskers represent the range of samples up to 1.5 times the interquartile range. Beyond this point, samples are shown as outliers. A. Quantification of CXCL10 mRNA (qTACT) pre vaccination and at 10 and 20 days post the first and second dose of an mRNA-based vaccine. Time points are indicated on the x-axis and relative CXCL10 expression (minus DMSO control) on the y-axis. The dashed line represents the qTACT threshold (0.05). The number of subjects for each time point is indicated above the box plots along with the percentage of subjects who fall above the threshold. Colors represent the subject's COVID-19/vaccination status (see legend). B. Quantification of IFNG mRNA (qTACT) pre vaccination and at 10 and 20 days post the first and second dose of an mRNA-based vaccine. Time points are indicated on the x-axis and relative IFNG expression (minus DMSO control) on the y-axis. The number of subjects for each time point is indicated above the box plots. Colors represent the subject's COVID-19/vaccination status (see legend). C. Quantification of IFN-γ protein secretion (ELLA) pre vaccination and at 10 and 20 days post the first and second dose of an mRNA-based vaccine. Time points are indicated on the x-axis and IFN-γ protein secretion (minus DMSO control) on the y-axis. The dashed line represents the ELLA threshold (5). The number of subjects for each time point is indicated above the box plots along with the percentage of subjects who fall above the threshold. Colors represent the subject's COVID-19/vaccination status (see legend). D. Quantification of IFN-γ producing cells (ELISpot) pre vaccination and at 10 and 20 days post the first and second dose of an mRNA-based vaccine. Time points are indicated on the x-axis and number of IFN-γ producing cells (minus DMSO control) on the y-axis. The dashed line represents the ELISpot threshold (5). The number of subjects for each time point is indicated above the box plots along with the percentage of subjects who fall above the threshold. Colors represent the subject's COVID-19/vaccination status (see legend).

FIGS. 6A-I. The figure provides data showing the use of dqTACT to monitor the persistence of cellular immunity and cross reactivity with spike epitopes from VOC in vaccinated subjects. For A-C and E-I, the box bounds represent the first quartile (bottom), median (center), and the third quartile (top). The whiskers represent the range of samples up to 1.5 times the interquartile range. Beyond this point, samples are shown as outliers. The number of subjects for each time point is indicated above the box plots along with the percentage of subjects who fall above the threshold. A-C. Detection of CXCL10 mRNA (dqTACT (A)), IFN-γ protein secretion (ELLA (B)), or IFN-γ producing cells (ELISpot (C)) in vaccinated subjects over time. Time points (post vaccination) are indicated on the x-axis and relative CXCL10 expression (A), IFN-γ protein secretion (B), or IFN-γ producing cells (C) on the y-axis (all values minus DMSO). The dashed lines represents the dqTACT (0.003), ELLA (5), or ELISpot (5) thresholds. D. Schematic to show how T cell responses against the variant of concern (VOC) can be evaluated using the delta variant as an example. Orange regions refer to amino acid mutations present in the delta variant compared to the wild-type (WT) SARS-CoV-2 strain. Pool HS contains peptides covering the non-conserved Spike-Wuhan regions affected by mutations present in the delta variant (24 peptides). Pool Delta MT contains peptides from Pool HS with the amino acid mutations present in the Spike protein of the delta variant. E-G. Quantification of CXCL10 mRNA (dqTACT (E)), IFN-γ protein secretion (ELLA (F)), or IFN-γ producing cells (ELISpot (G)) in vaccinated subjects stimulated with spike peptides covering the hotspot wildtype (HS WT) or delta variant region. The peptide pool used for stimulation, either the wildtype (HS) or delta (delt) sequence, is indicated on the x-axis and relative CXCL10 expression (E), IFN-γ protein secretion (F), or IFN-γ producing cells (G) on the y-axis (all values minus DMSO). The dashed lines represents the dqTACT (0.003), ELLA (5), or ELISpot (5) thresholds. P-values were calculated using a two-sided Wilcoxon rank sum method. H-I. Quantification of relative CXCL10 mRNA expression using dqTACT (H) or IFN-γ protein section using ELLA (I) in an elderly cohort.

FIGS. 7A-E. The figure shows that CXCL10 mRNA levels can be measured as a proxy for T cell activation.

FIGS. 8A-B. The figure shows that CXCL10 mRNA is a reliable proxy for IFN-γ secreted by antigen specific T cells.

FIGS. 9A-E. The figure shows a significant correlation between TACTseq and either ELLA or ELISpot.

FIGS. 10A-E. The figure shows that stimulation with spike (SpG), but not NP2 peptides nor DMSO, induce the selective induction of CXCL10/IP-10, CCL2, CCL4, CCL8, CXCL8 and CXCL9 in vaccinated subjects.

FIGS. 11A-C. The figure shows that COVID-19 recovered individuals had a higher median expression of IFNG prior to vaccination.

### DETAILED DESCRIPTION

The present disclosure provides a rapid and internally normalized qPCR-based assay for detection of virus specific cellular immunity that avoids the need for cell lysis and RNA purification, and that is rapid, scalable, and accurate. In an embodiment, the assay detects SARS-CoV-2 cellular immunity.

The present disclosure provides methods for detecting, staging, and treating a viral infection, including SARS-CoV-2, using a qPCR-based assay where the assay can quantify T cell activation by said viral antigens. The present disclosure provides methods for determining whether to and when to vaccinate a subject, including a vaccination against SARS-CoV-2.

The present disclosure provides a rapid, user-friendly, accessible, scalable, and accurate diagnostic method to quantify cellular immunity against viruses, including SARS-CoV-2. The present qPCR-based dqTACT assay is amenable to periodic and repeated testing of patient samples, as it requires only 1 ml of blood and provides a 24-hour turnaround time.

The present disclosure provides a derived profile of SARS-CoV-2-specific T cell activation using qTACT/dqTACT assays in different cohorts of naïve, COVID-19 recovered, and vaccinated individuals, and it includes robust information about the level of SARS-CoV-2-specific cellular immunity in those individuals. The present invention can be easily adapted to detect the degree of cellular immunity is an urgently needed complement to the currently available tests measuring viral presence or antibody titers, and design future vaccination strategies according to the levels of immune protection in the population.

### EXAMPLES

The following examples are presented to provide those of ordinary skill in the art with a complete disclosure and description of the assaying, screening, and therapeutic methods of the invention, and are not intended to limit the scope of the invention.

### Example 1

Ex-Vivo Stimulation of Whole Blood with SARS-CoV-2 Viral Proteins

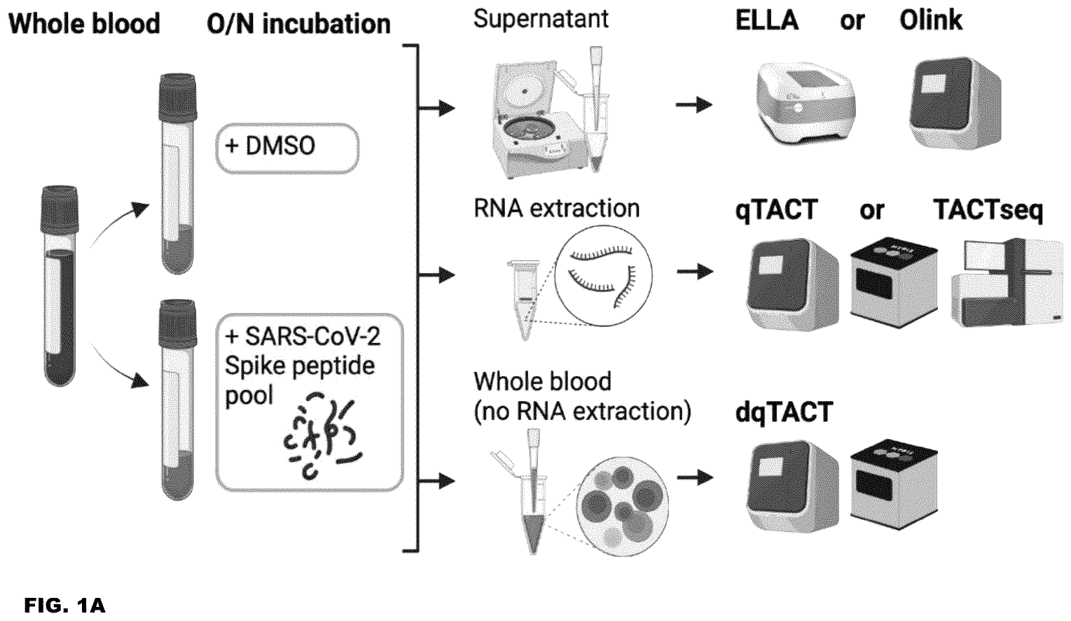

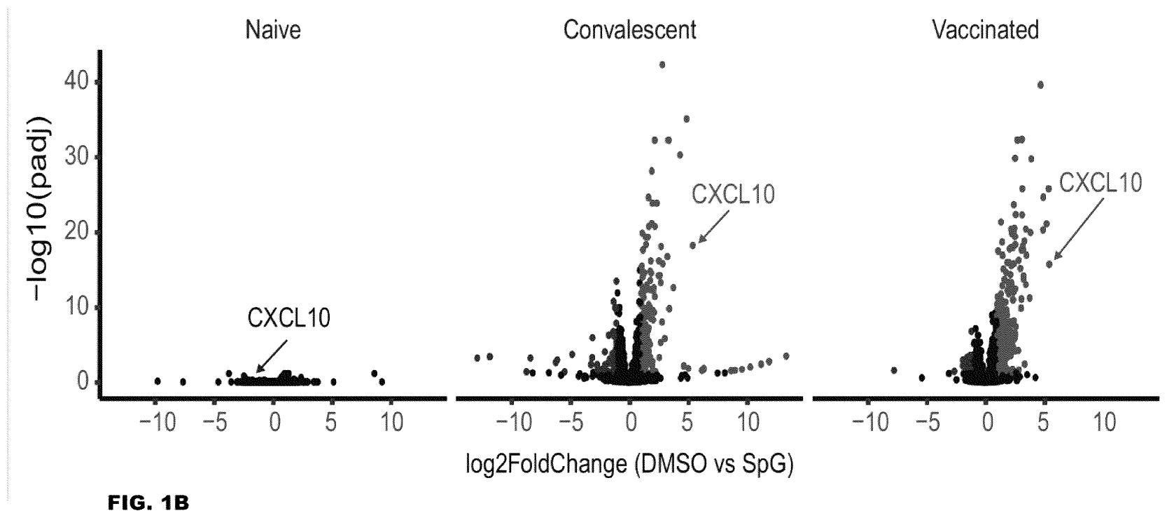

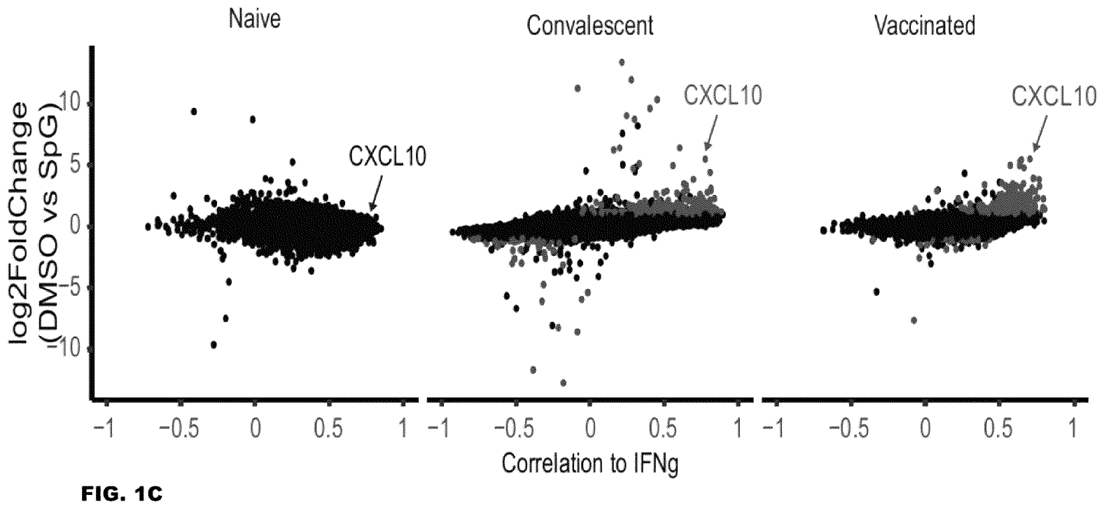

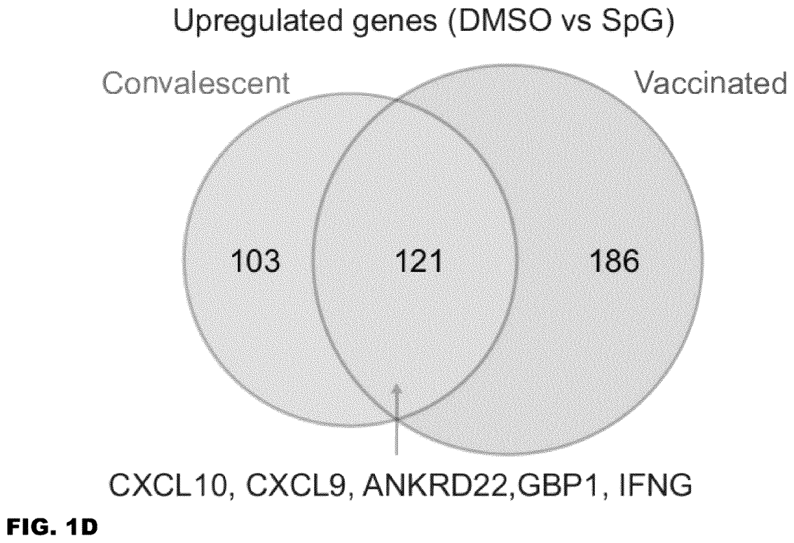

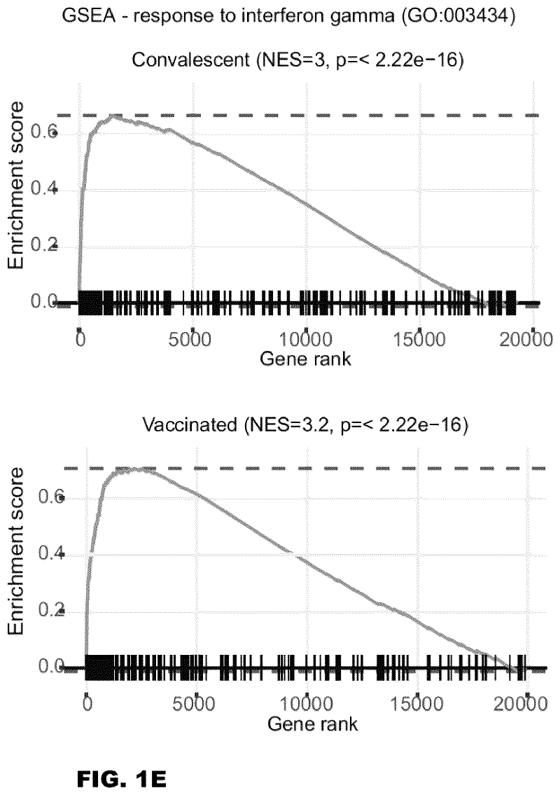

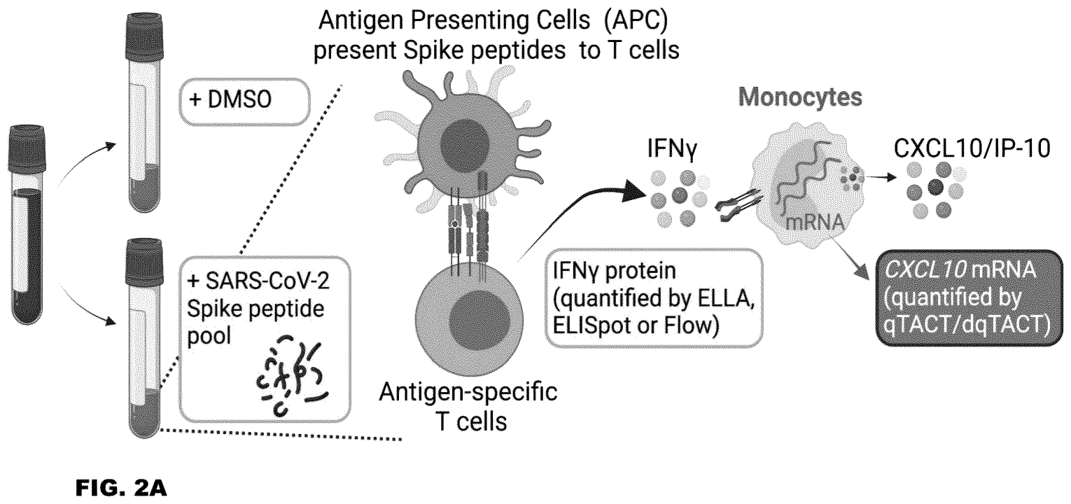

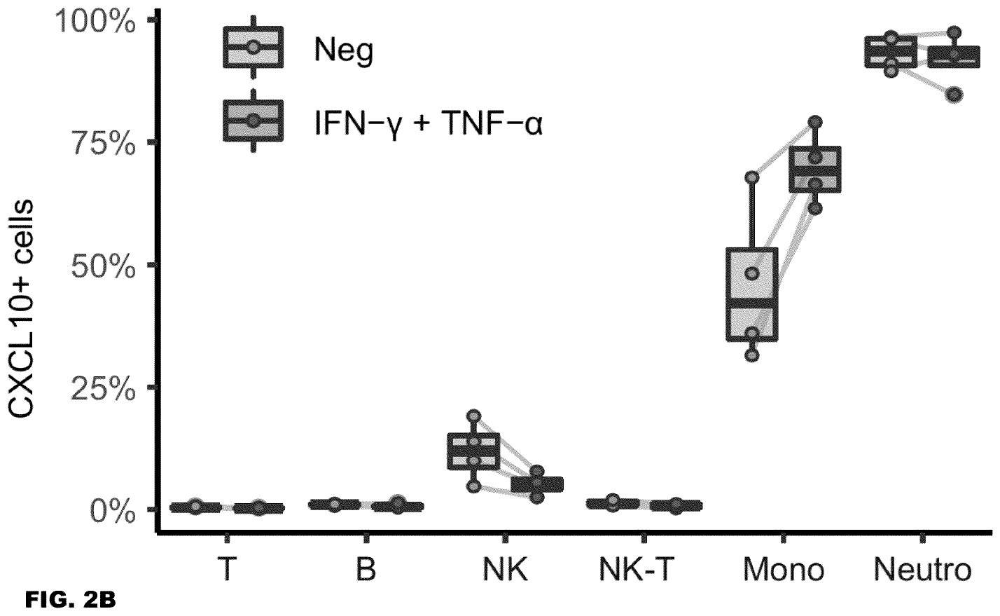

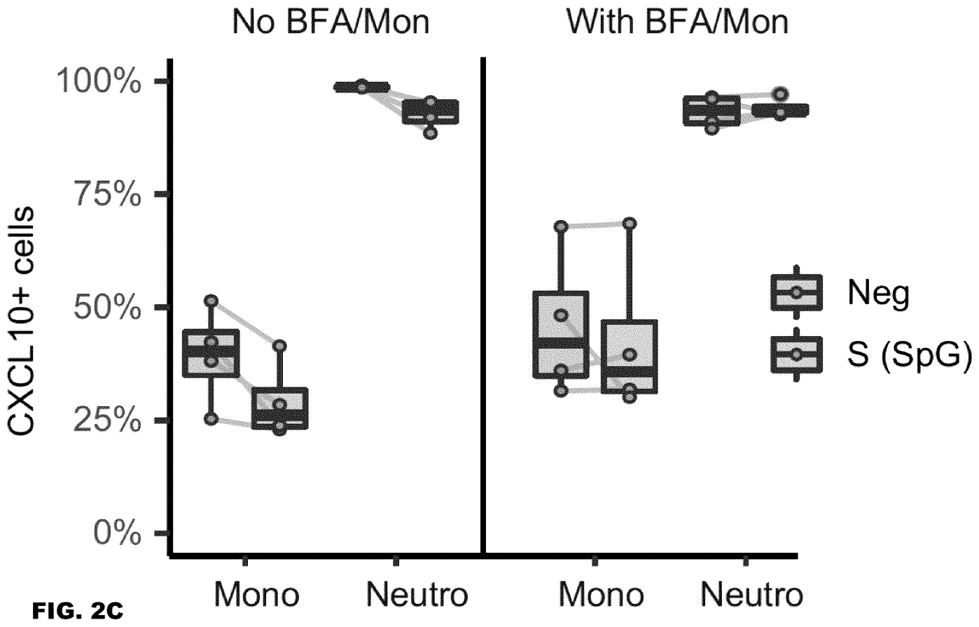

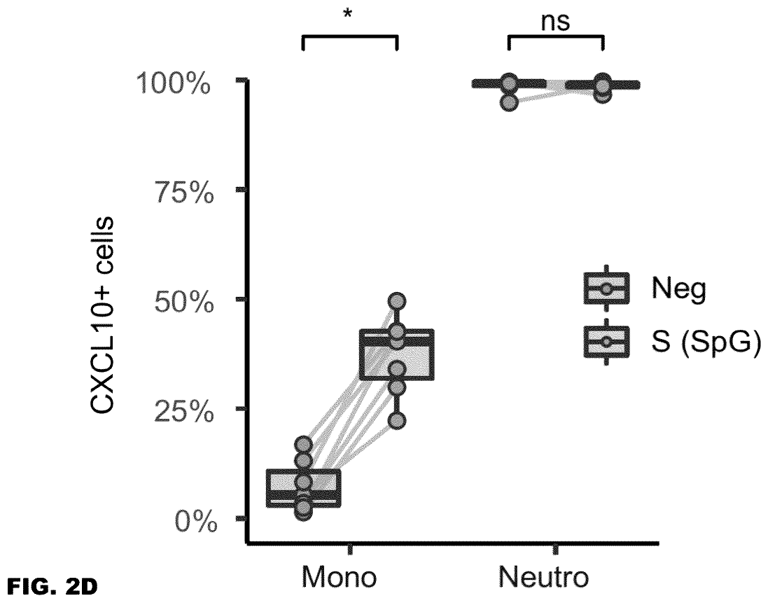

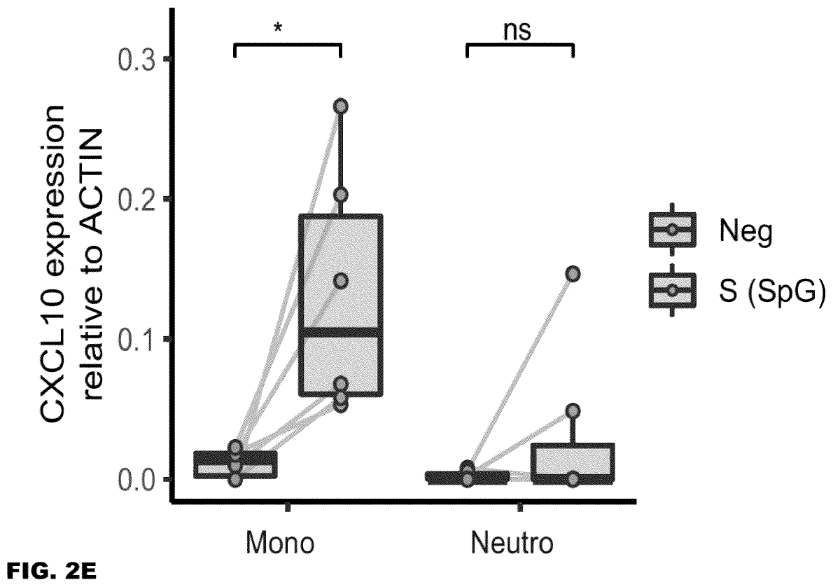

The inventors have implemented a probe-based qPCR rapid T cell activation (qTACT) assay (FIG. 1A), based on ex vivo stimulation of whole blood samples with a pool of viral peptides covering spike [S] or other SARS-CoV-2 viral proteins (i.e. nucleoprotein [NP]), followed by direct amplification of IFNG (directly produced by SARS-CoV-2 antigen-specific T cells) or CXCL10, a molecule expressed by monocytes in response to T cell activation (FIG. 2A). A further technical implementation of the assay allows quantification of T cell immunity directly from blood, bypassing the need for red blood cell (RBC) lysis and RNA purification, thus reducing labor and time and minimizing operator-induced errors. To this end, following overnight incubation with a DMSO control, or SARS-CoV-2 peptide pools, 50 microliters of blood are diluted (1:4) to avoid PCR inhibition by anticoagulants (i.e. heparin), and 2 microliters are directly loaded onto a qPCR instrument (dqTACT). This latter assay is referred to as direct qPCR-based rapid T cell activation (dqTACT) (FIG. 1A).

To select genes whose induction would correlate with the presence and activation of antigen-specific T cells, the inventors first evaluated the transcriptional profile of whole blood after overnight stimulation with SARS-CoV-2-peptide pools by RNA-sequencing (TACTseq, FIG. 1A). This initial cohort consisted of 11 naïve, 8 COVID-19 recovered subjects and 16 vaccinated subjects (13 collected at 2-3 months and 5 collected at 5-8 months after the second BNT162b2 dose).

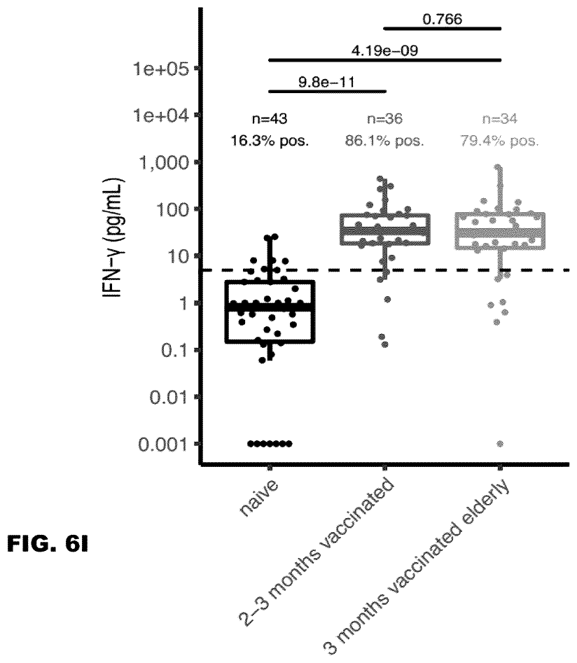

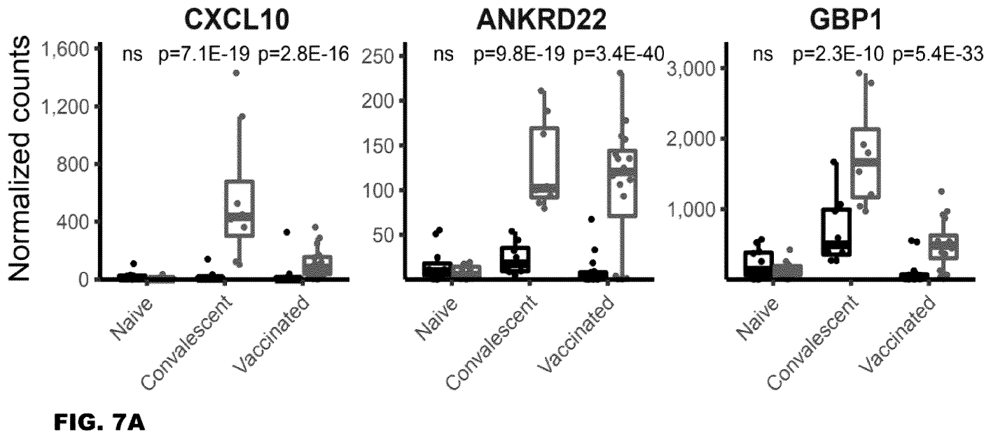

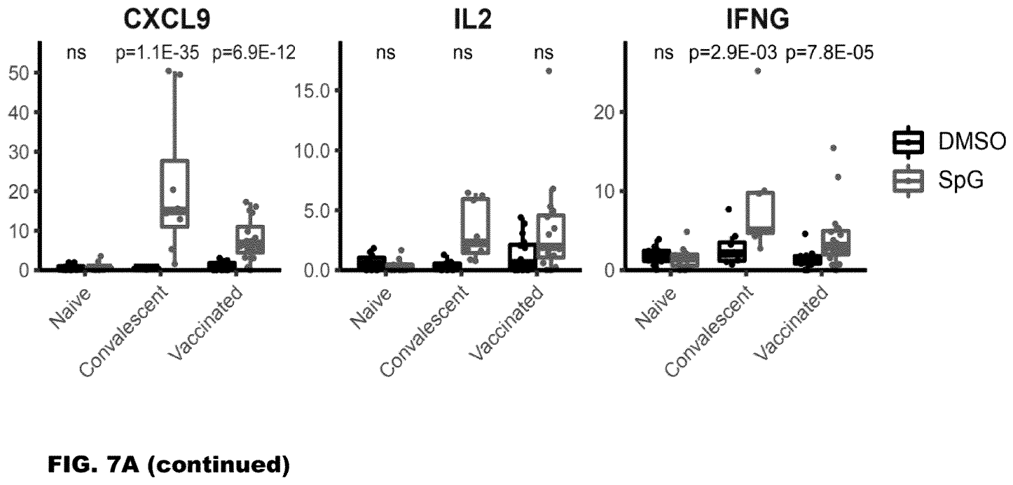

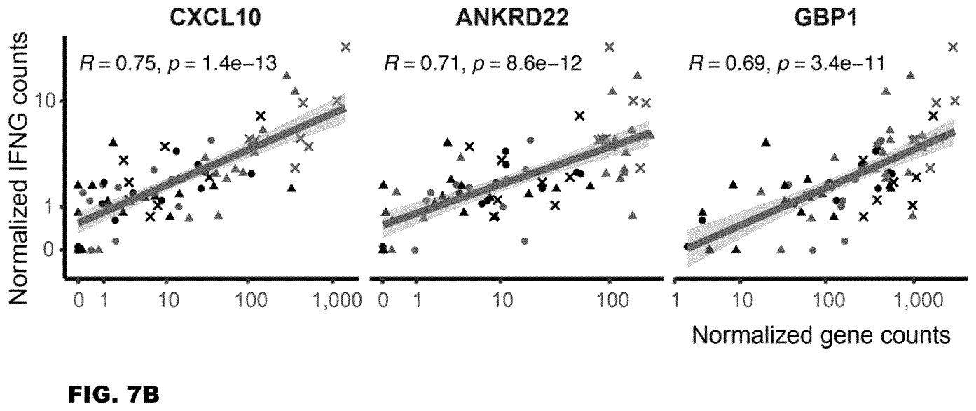

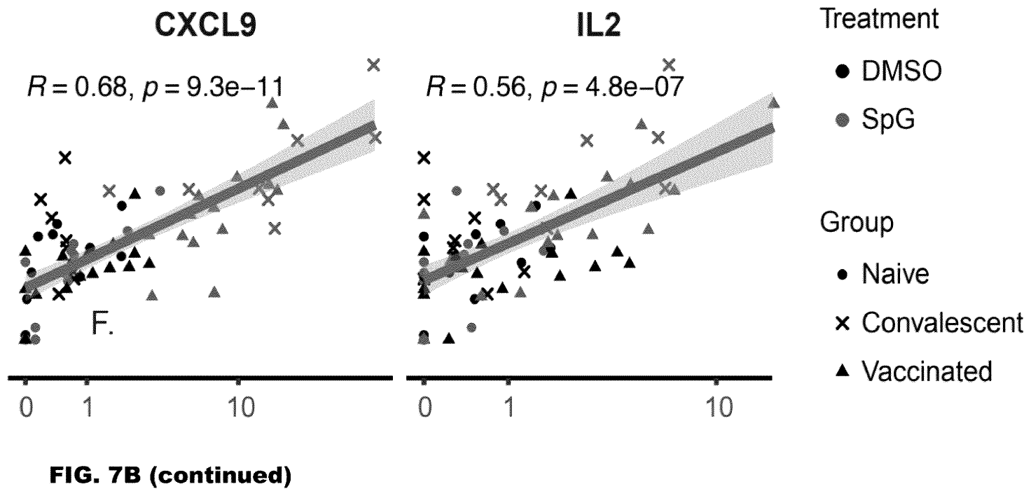

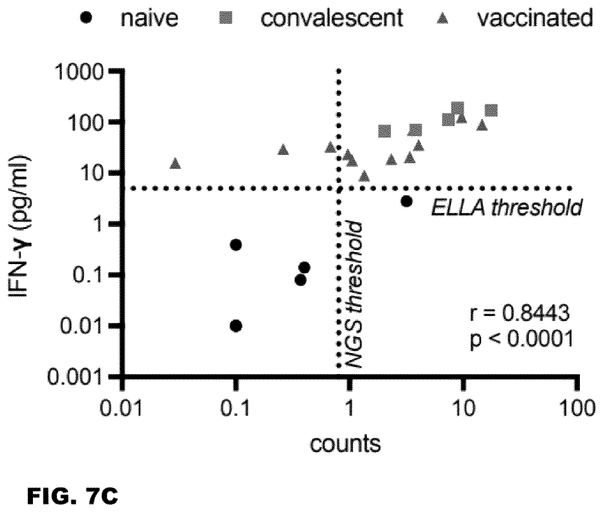

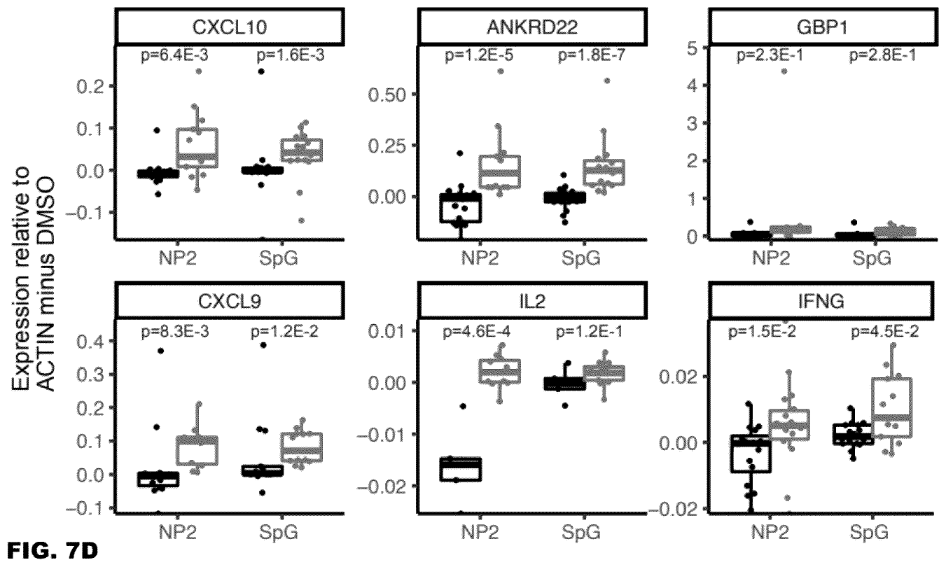

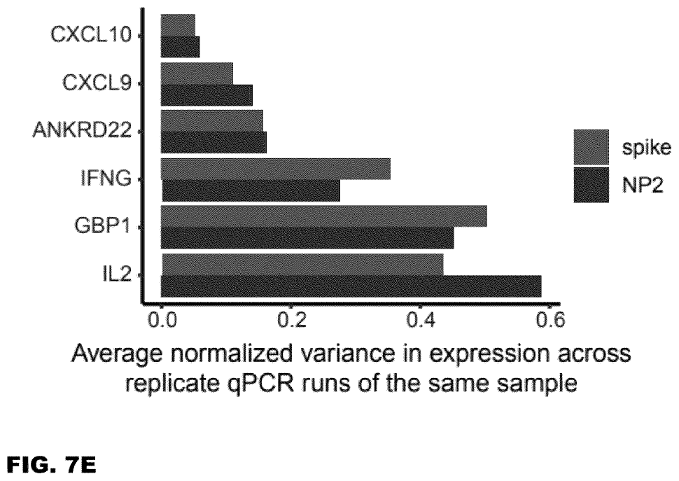

Briefly, whole blood was incubated overnight with either DMSO or Spike-Gold (SpG) peptides. The latter is a refined set of peptides covering immunodominant spike peptides (previously described and validated in Le Bert, N. et al. (2021)). RNA was extracted from the cell pellet and subjected to Illumina single-end sequencing (Koh, C. M. et al. (2015)). The inventors identified genes activated by viral peptides by performing a differential expression analysis between peptide-stimulated samples and untreated controls, across all subjects (FIG. 1B). Treatment with SpG robustly induced a largely overlapping set of genes (121 genes, FDR<0.05, log 2FoldChange>1) in both SARS-CoV-2 convalescent and vaccinated subjects (FIG. 1D). Genes associated with the “cellular response to interferon gamma signaling” pathway are significantly enriched among these upregulated genes, consistent with antigen-specific T cell activation in convalescent or vaccinated subjects (FIG. 1D). To narrow down a shortlist of candidates for further investigation by qPCR, the inventors selected a panel of the top 6 genes that were significantly upregulated following SpG stimulation (FIG. 1E) and that, importantly, correlated with IFNG expression, as a proxy for antigen specific T cell activation (FIG. 1C, FIG. 7B). Of note, correlation between IFNG mRNA levels and IFN-γ protein (detected by ELLA), were high, supporting the initial screen (FIG. 7C).

### Example 2

### CXCL10 Induction in Monocytes

The inventors validated transcriptional induction of shortlisted genes by qPCR in 13 COVID-19 convalescent and 16 naïve subjects. This included stimulation with a refined sets of peptides covering immunodominant SARS-CoV-2 nucleoprotein peptides [NP2], in addition to the SpG pool (Le Bert, N. et al. (2021); Kalimuddin, S. et al. (2021)). The former was used as an additional control to differentiate between naïve and convalescent subjects. For each gene, the same subject was run between 2 to 10 times to determine how results varied with multiple replicates. Out of all genes tested (FIG. 7D), CXCL10 displayed the lowest variance (FIG. 7E), and is thus the most reproducible and reliable biomarker for the PCR-based assay.

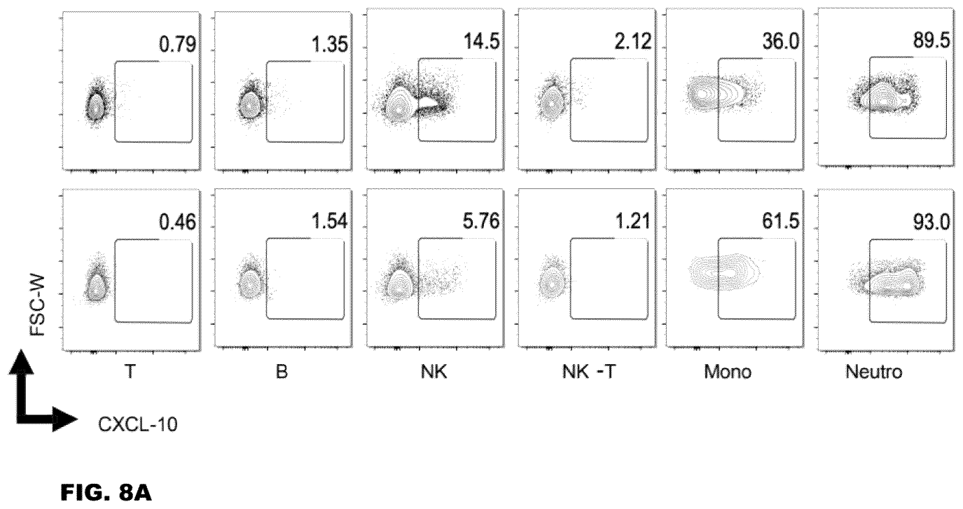

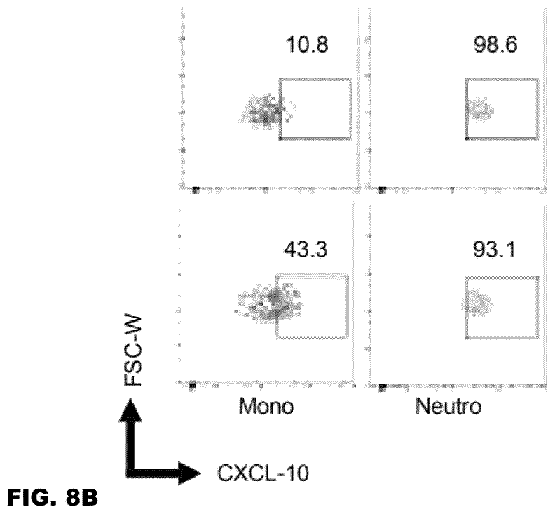

The inventors hypothesized that CXCL10 mRNA is a reliable proxy for IFN-γ secreted by antigen specific T cells (FIG. 2A). CXCL10/IP-10 protein is less so (Petrone, L. et al. (2021), as it is abundantly stored by neutrophils and monocytes prior to IFN-γ stimulation. To prove this, the inventors performed the following analysis: first, they demonstrated which immune cell subsets produce CXCL10/IP-10 in response to stimulation with IFN-γ and TNF-α in the presence of brefeldin/monensin (BFA/Mon) to inhibit protein secretion. Monocytes and neutrophils are the main immune cells that increase their CXCL10/IP-10 production in response to IFN-γ and TNF-α stimulation. Monocytes and neutrophils, in particular, have elevated CXCL10/IP-10 levels at baseline (before stimulation) (FIG. 2B, FIG. 8A).

Second, the inventors determined whether monocytes and neutrophils produce CXCL10/IP-10 upon stimulation with SARS-CoV-2 specific spike peptides. For this purpose, the inventors set up three conditions: (1) BFA/Mon was not added, allowing cytokine release from immune cells and (2) a negative control in which BFA/Mon was added from the beginning blocking IFN-γ production and CXCL10 mRNA induction in neighboring cells (FIG. 2C); and (3) BFA/Mon was added in the last 4 hours of an overnight incubation (delayed BFA/Mon), which prevents CXCL10/IP-10 secretion in the cell culture media, but should not prevent CXCL10 mRNA induction (FIG. 2D, FIG. 8B). The flow cytometric results indicated an accumulation of CXCL10/IP-10 in monocytes and not in neutrophils upon stimulation with spike peptides (FIG. 2C-D). Third, and significantly, the inventors sorted cells in the BFA/Mon delayed condition; mRNA was extracted, and CXCL10 levels were quantified by qTACT (FIG. 2E), confirming a significant induction only in monocytes.

The inventors determined that monocytes can produce CXCL10 in a tightly regulated manner and in response to the IFN-γ secreted by antigen-specific T cells. Thus, they discovered that this signal serves as a proxy of T cell activation upon spike peptide stimulation of whole blood (FIG. 2A).

### Example 3

CXCL10 mRNA Expression Correlates with the IFN-γ Level

The inventors assessed the correlation between CXCL10 mRNA expression and IFN-γ level in experiments using a larger cohort of naïve, COVID-19 convalescent and SARS-CoV-2 vaccinated subjects.

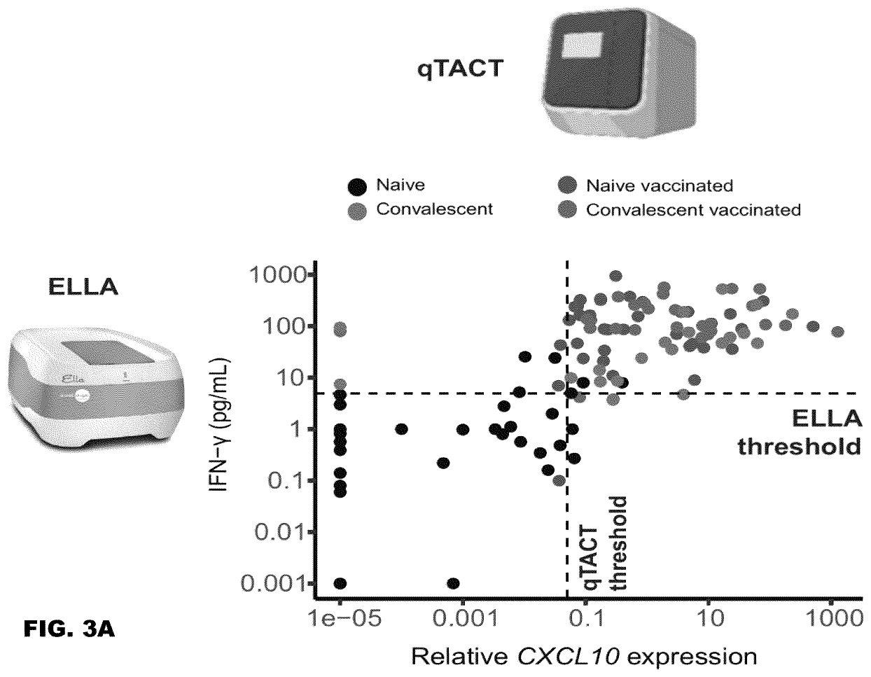

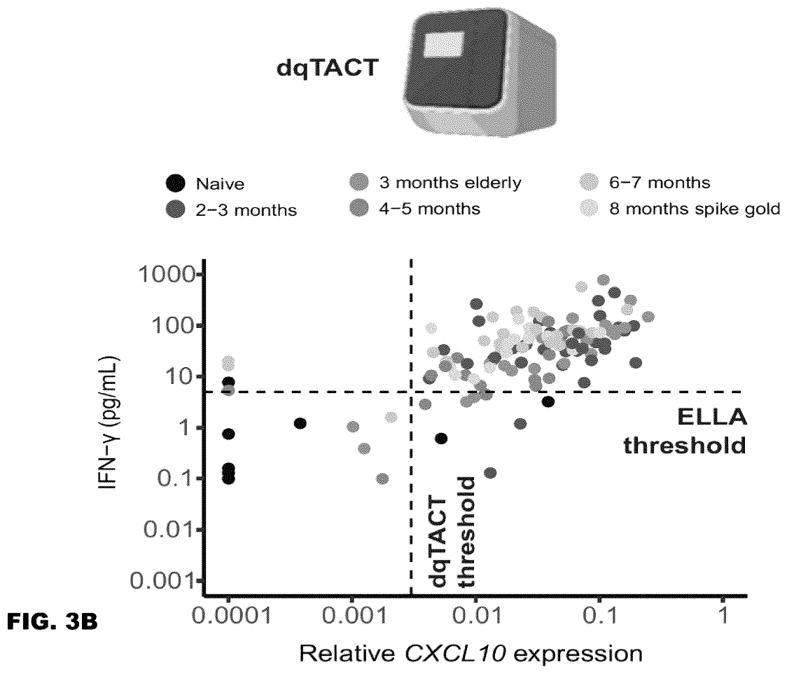

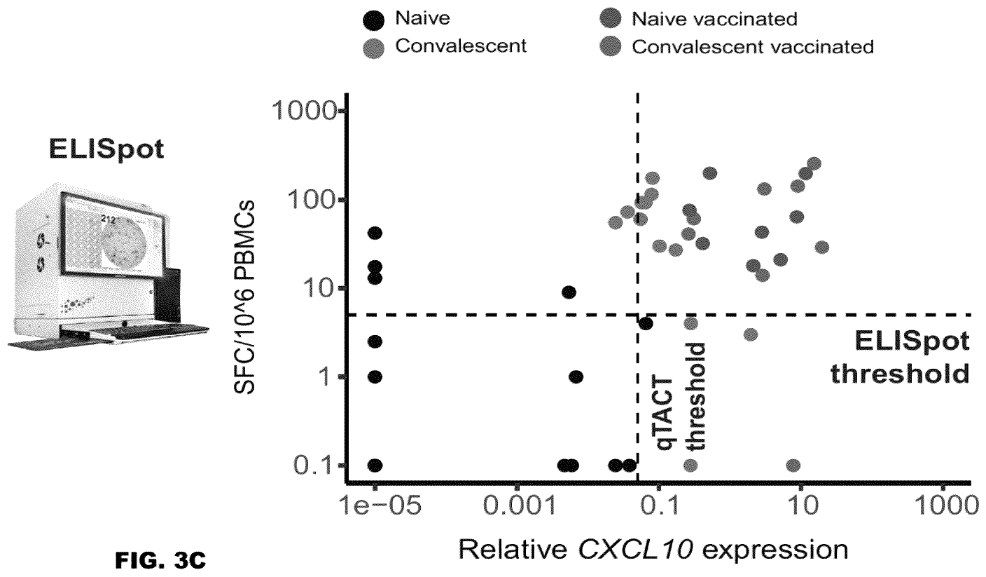

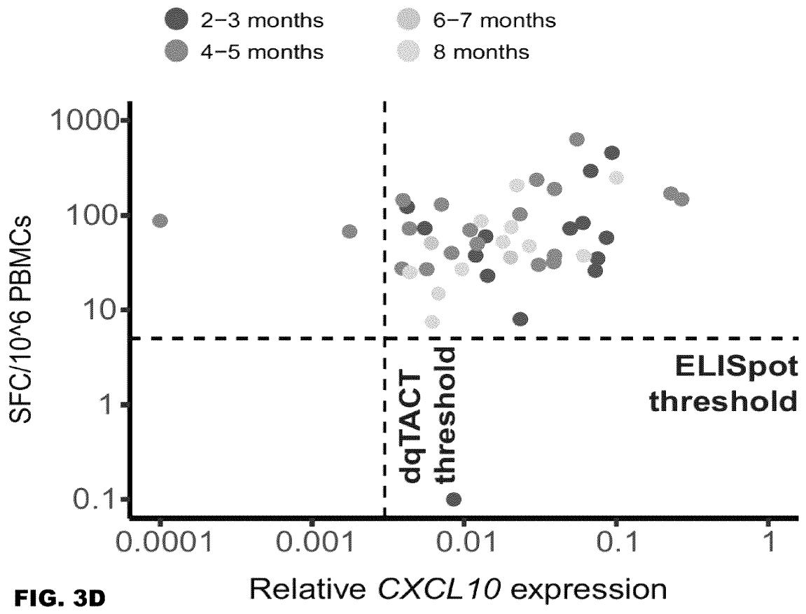

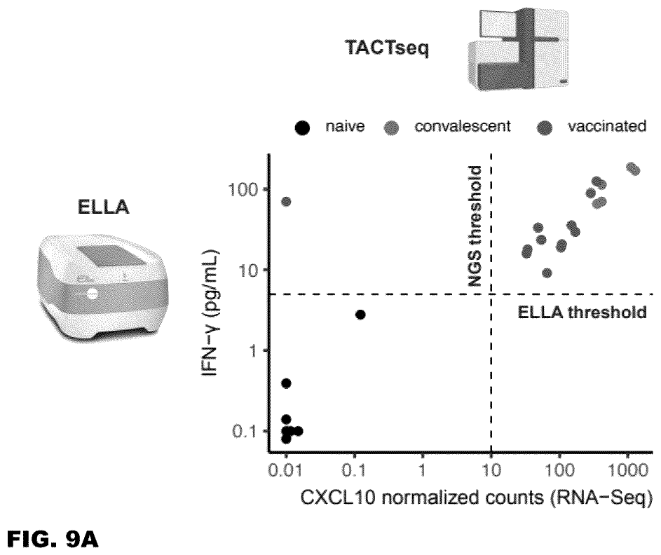

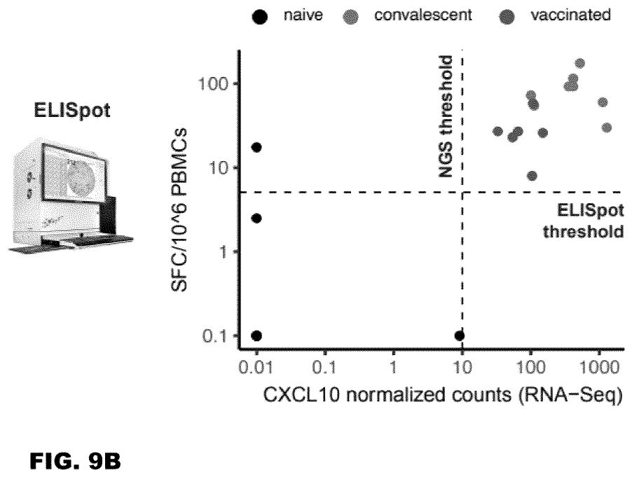

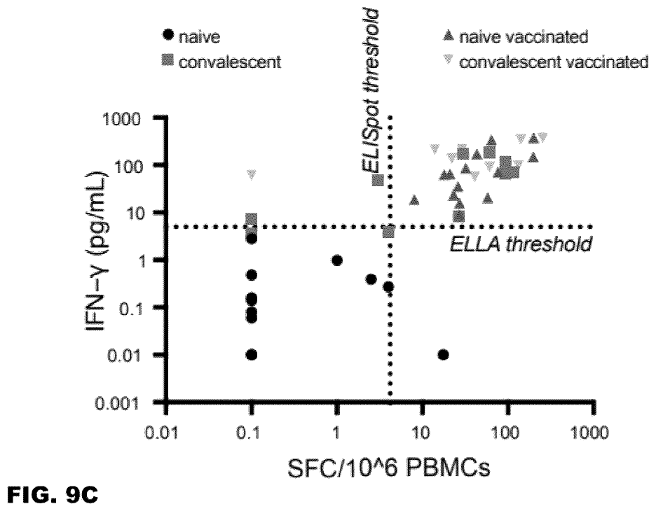

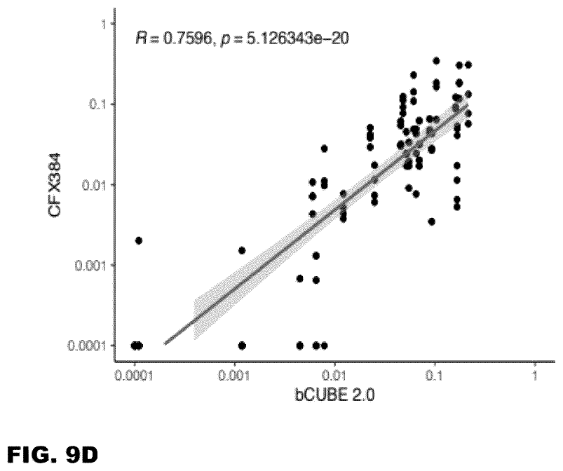

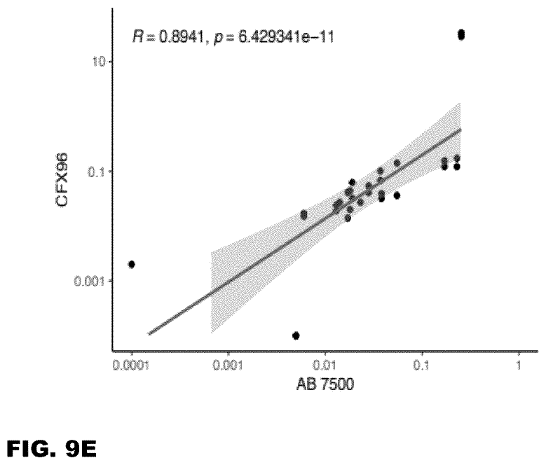

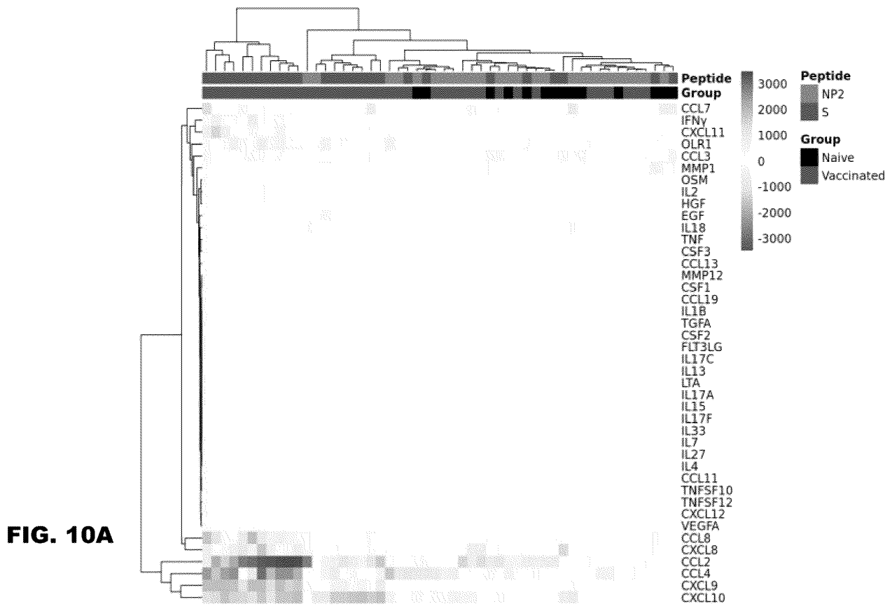

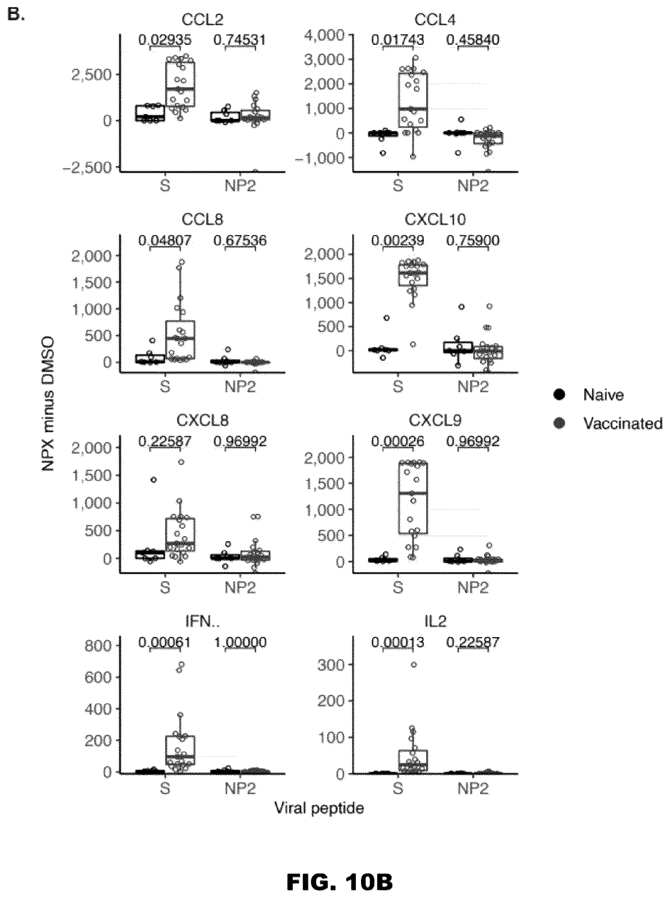

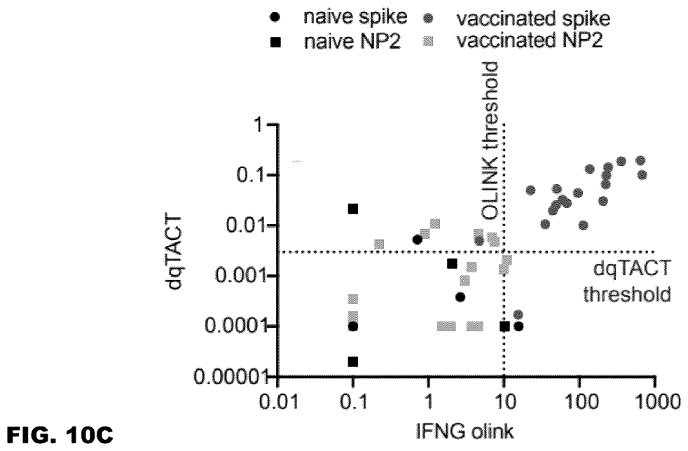

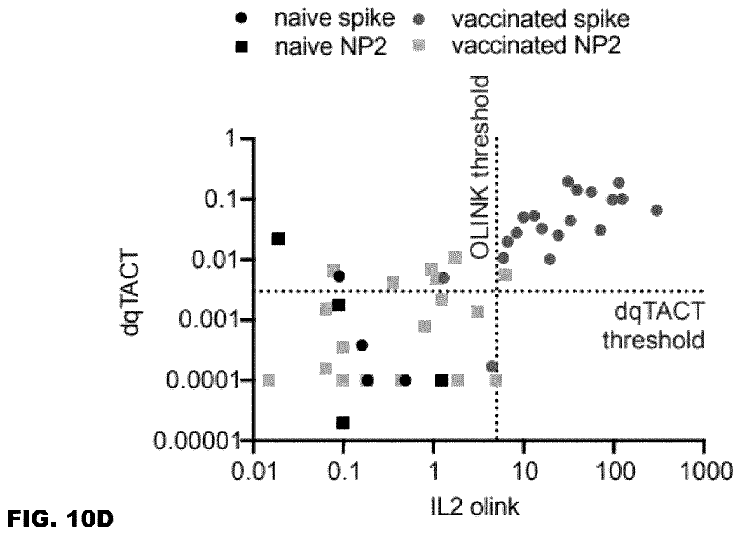

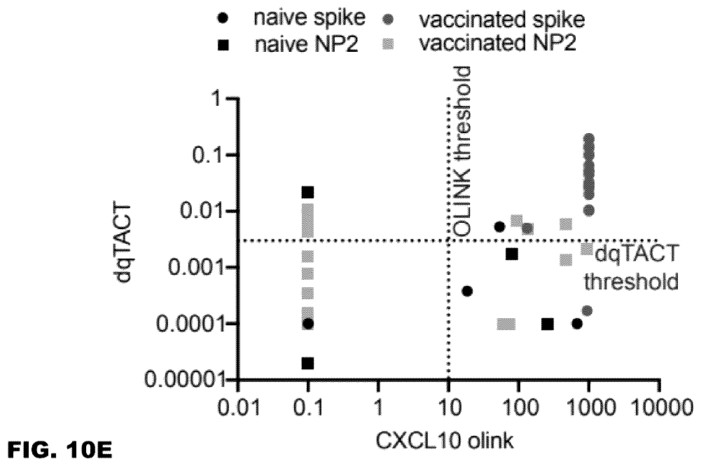

The inventors discovered that CXCL10 measured by both qTACT and dqTACT is in significant concordance with both IFN-γ protein quantification by ELLA (FIG. 3A-B; Tables 1 & 2) and ELISpot (FIG. 3C-D; Tables 3 & 4). The inventors additionally demonstrated a significant correlation between TACTseq and either ELLA or ELISpot (FIG. 9A-B). In addition to TACTseq, the inventors used an independent and complementary approach to assess the cytokines/chemokines induced by both lymphoid and myeloid cells in whole blood. Following stimulation with SARS-CoV-2 peptide pools, the supernatant of naïve (n=7) and BNT162b2 vaccinated (n=19) subjects was collected and analyzed using the Olink multiplex assay (Olink Bioscience, Uppsala, Sweden). This technology can accurately quantify secretion of a panel of 45 inflammatory cytokines/chemokines. Surprisingly, stimulation with spike (SpG), but not NP2 peptides nor DMSO, induced the selective induction of CXCL10/IP-10, CCL2, CCL4, CCL8, CXCL8 and CXCL9 in vaccinated subjects (FIG. 10 A-B) (in addition to stimulation of IFN-γ and IL2).

TABLE 1

| | |
|---|---|
| Average age | 45.15 |
| Age range | 22-76 |
| Percentage female (%) | 69.4 |
| Naïve N | 34 |
| Convalescent N | 21 |
| Naïve vaccinated N | 35 |
| Convalescent vaccinated N | 27 |
| Total N | 117 |

| | Average | | Average IFN- | |
|---|---|---|---|---|
| | CXCL10 | Standard | γ secretion | Standard |
| | expression | deviation | (pg/mL) | deviation |
| Naive | 0.03 | 0.07 | 2.93 | 5.98 |
| Convalescent | 22.45 | 52.24 | 82.32 | 124.29 |
| Naive vaccinated | 20.57 | 86.08 | 149.46 | 175.46 |
| Convalescent | 65.02 | 246.52 | 201.51 | 162.99 |
| vaccinated | | | | |

TABLE 2

| | |
|---|---|
| Average age | 42.73 |
| Age range | 17-81 |
| Percentage female (%) | 46.9 |
| Naïve N | 9 |
| 2-3 months vaccinated N | 35 |
| 3 months vaccinated (elderly) N | 23 |
| 4-5 months vaccinated N | 19 |
| 6-7 months vaccinated N | 24 |
| 8 months vaccinated N | 14 |
| N | 124 |

| | | Average | | Average IFN- | |
|---|---|---|---|---|---|
| | | CXCL10 | Standard | γ secretion | Standard |
| | | expression | deviation | (pg/mL) | deviation |
| Naive | | 0.005 | 0.013 | 1.56 | 2.525 |
| 2-3 | months | 0.063 | 0.054 | 70.365 | 93.388 |
| 3 | months elderly | 0.074 | 0.066 | 92.347 | 165.634 |
| 4-5 | months | 0.027 | 0.024 | 27.852 | 33.383 |
| 6-7 | months | 0.035 | 0.037 | 96.021 | 117.055 |
| 8 | months | 0.027 | 0.026 | 51.048 | 36.573 |

TABLE 3

| | |
|---|---|
| Average age | 37.09 |
| Age range | 24-62 |
| Percentage female (%) | 47.1 |
| Naïve N | 20 |
| Convalescent N | 13 |
| Naïve vaccinated N | 8 |
| Convalescent vaccinated N | 9 |
| Total N | 50 |

| | Average | | Average | |
|---|---|---|---|---|
| | CXCL10 | Standard | SFC/10\hat{ }6 | Standard |
| | expression | deviation | PBMCs | deviation |
| Naive | 0.01 | 0.02 | 4.56 | 10.08 |
| Convalescent | 0.25 | 0.52 | 55.9 | 53.13 |
| Naive vaccinated | 3.96 | 4.24 | 81.25 | 74.74 |
| Convalescent vaccinated | 7.31 | 7.18 | 84.39 | 86.68 |

TABLE 4

| | |
|---|---|
| Average age | 30.53 |
| Age range | 17-60 |
| Percentage female (%) | 62.5 |
| 2-3 months vaccinated N | 14 |
| 4-5 months vaccinated N | 19 |
| 6-7 months vaccinated N | 2 |
| 8 months vaccinated N | 11 |
| N | 46 |

| | | Average | | Average | |
|---|---|---|---|---|---|
| | | CXCL10 | Standard | SFC/10\hat{ }6 | Standard |
| | | expression | deviation | PBMCs | deviation |
| 2-3 | months | 0.042 | 0.033 | 96.436 | 126.761 |
| 4-5 | months | 0.043 | 0.075 | 121 | 139.029 |
| 6-7 | months | 0.013 | 0.01 | 43.5 | 10.607 |
| 8 | months | 0.026 | 0.029 | 75.636 | 79.976 |

Surprisingly, the inventors discovered that qTACT CXCL10 quantification correlates well with Olink IFN-γ and IL-2 levels, and less so with CXCL10/IP-10. This is consistent with the finding that CXCL10 mRNA but not protein is the best proxy for T cell activation (FIG. 10C-E). This discrepancy is most likely due to the pre-stimulation reservoir of CXCL10/IP-10 in neutrophils (FIG. 2C), which are highly abundant in whole blood (Hsu, A. Y. et al. (2021)). Finally, the inventors demonstrated a high degree of correlation between ELLA and ELISpot quantified IFN-γ (FIG. 9C).

### Example 4

### Establishing Sensitivity and Specificity Thresholds of the Assay Using Various Machines

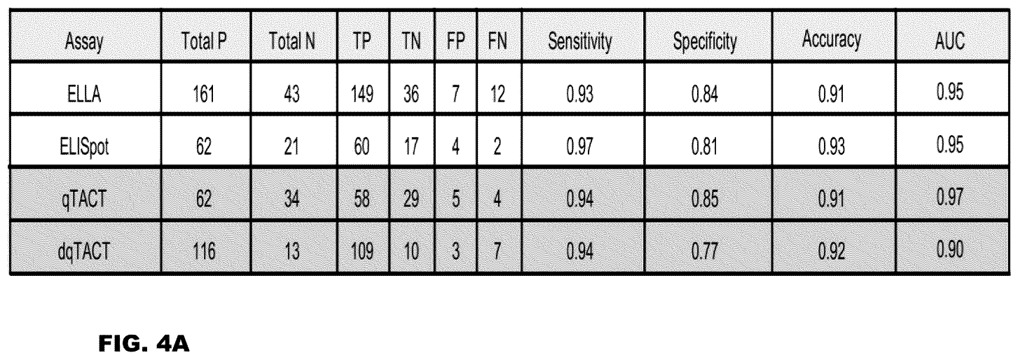

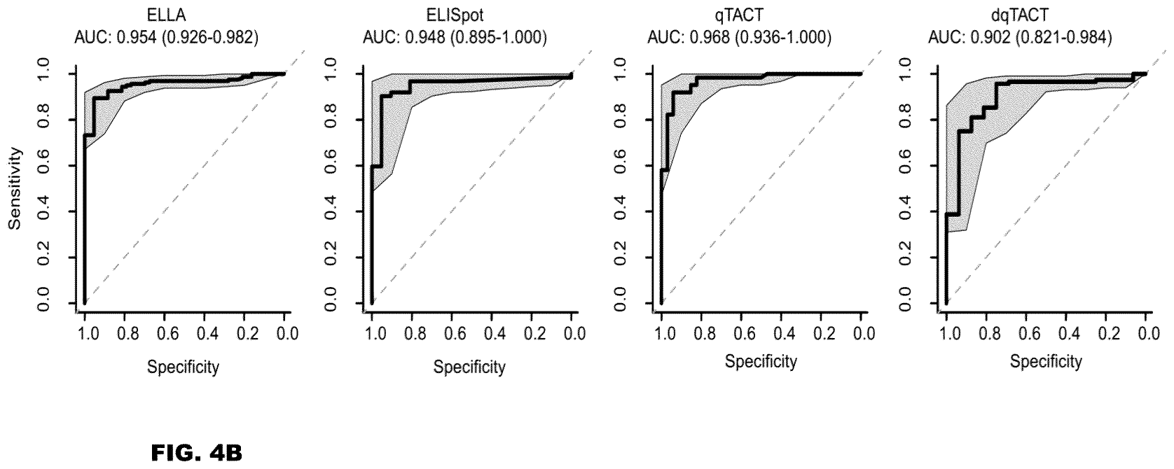

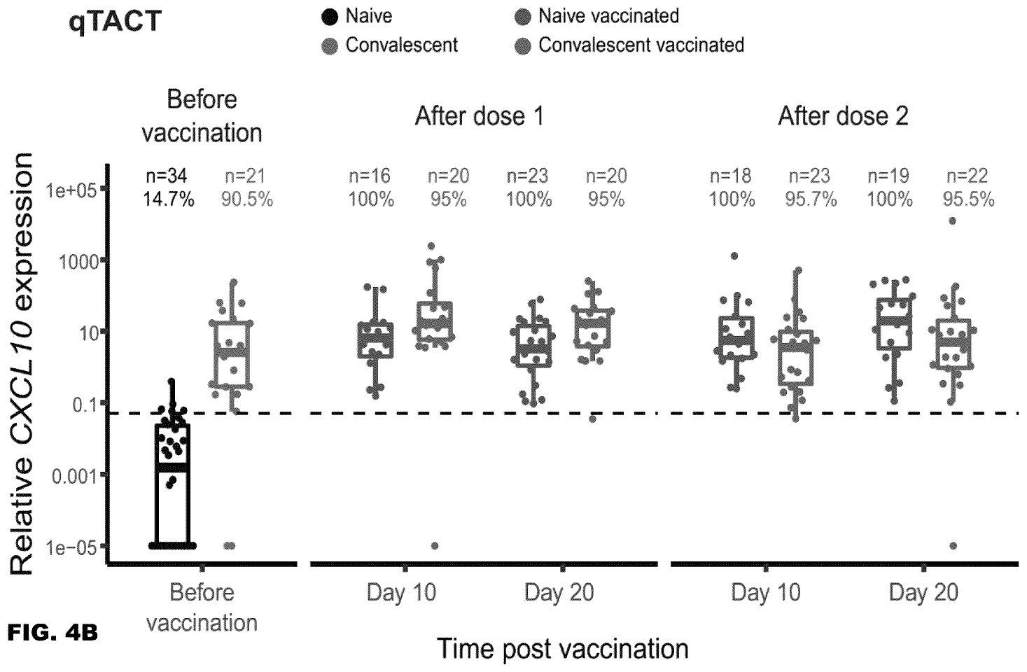

Each method has a different dynamic range, but the inventors were able to use their large set of data to establish robust thresholds to call true positives and true negatives, and to assess specificity, sensitivity, and accuracy of the different assays. The inventors also performed standard receiver operating characteristic (ROC) curve analysis to calculate the area under the curve (AUC). Overall, each assay has high accuracy and AUC >0.90 (FIG. 4A-B). The inventors additionally computed two-sided Fisher's exact tests for each assay, obtaining the associated p-values and odds ratios (with corresponding 95% CIs) in Table 5. These additional metrics confirm the high standards of the qTACT and dqTACT tests, which are comparable to the gold standard ELLA and ELISpot assays. These assays will be useful for the evaluation of cellular immunity towards future vaccines and variants, each with their advantages and limitations.

TABLE 5

| | p-value | Odds ratio |
|---|---|---|
| Assay | (Fisher's exact test) | (95% CI) |
| ELLA | 9.52e−23 | 61.1 (21.5-200.2) |
| ELISpot | 4.46e−12 | 109.8 (18.1-1312.5) |
| qTACT | 2.73e−15 | 76.3 (18.2-432.8) |
| dqTACT | 2.10e−09 | 43.4 (10.2-236.3) |

Traditional flow cytometry and ELISpot have the highest specificity and sensitivity, but they are difficult to scale given the intense labor time needed to isolate PBMCs. Whole blood-based assays are very comparable in terms of specificity, sensitivity, and accuracy. The strongest downside of ELLA, which directly quantifies IFN-γ secretion, is the price per sample, with the additional requirement of specialized equipment and/or plates. Finally, both qTACT and dqTACT were optimized on multiple qPCR machines, including the 7500 Fast System (Applied Biosystems), CFX96 (BioRad), CFX384 (BioRad), and bCUBE 2.0 (Hyris), with comparable results (Supplementary FIG. 3D-E), thus allowing a wide application of these studies across multiple diagnostic labs worldwide. The Hyris bCUBE is a portable, 2-channel qPCR machine that can quantify up to 36 samples at a time. Unlike BioRad's CFX machines, the bCUBE uses a patented detection technology based on a solid-state CMOS sensor with a peculiar optical stack to capture and quantify fluorescence, coupled with a disposable cartridge specifically designed for this setup. It features a reaction chamber design with reagents lying directly on the optical window, in conjunction with the efficient heat transfer given by an aluminum plate integrated in the cartridge. Importantly, as opposed to the BioRad or AB machines, this is a portable, inexpensive, and easy to use instrument that might enable point of care implementation of the assay.

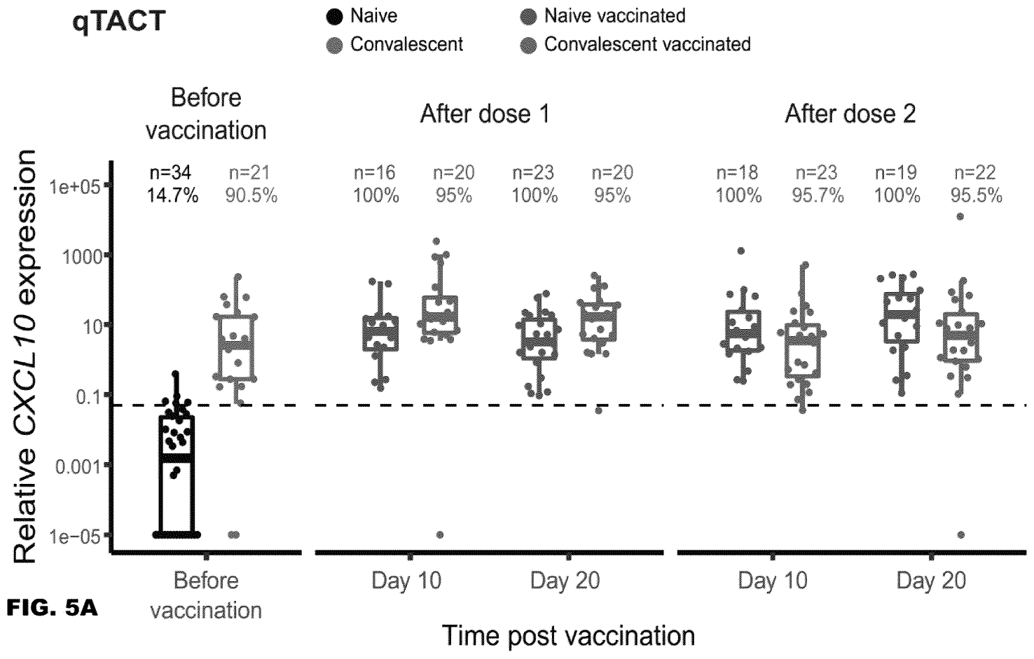

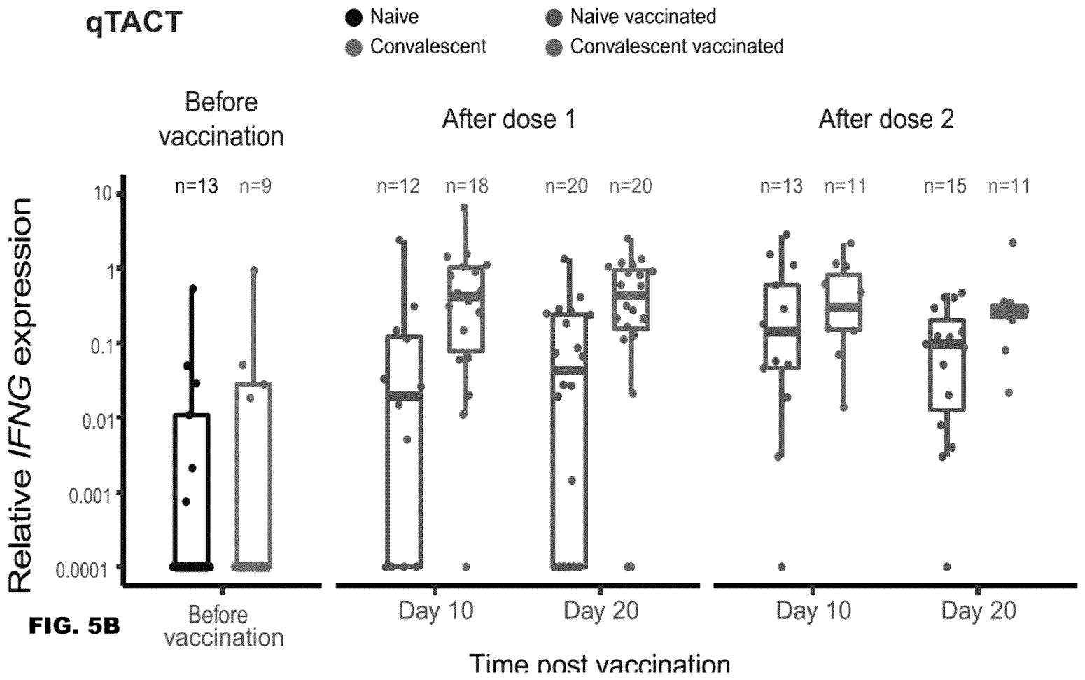

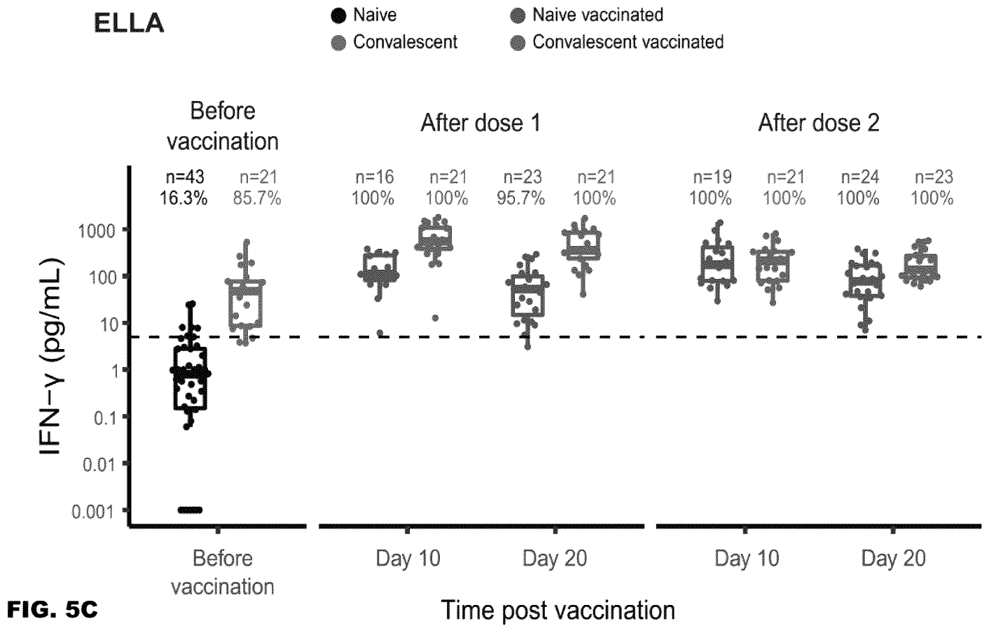

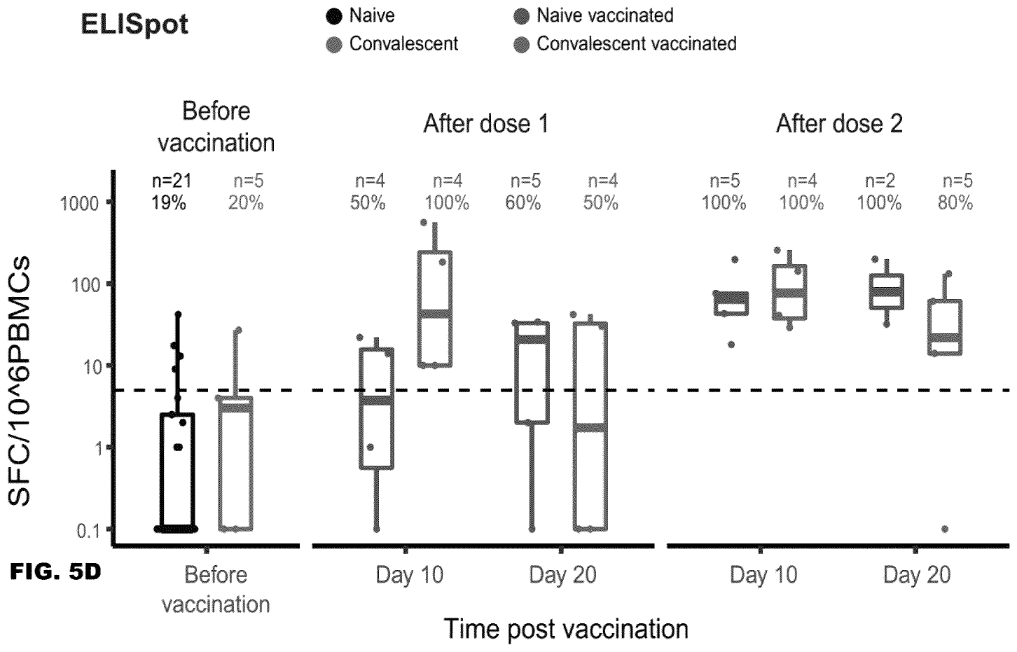

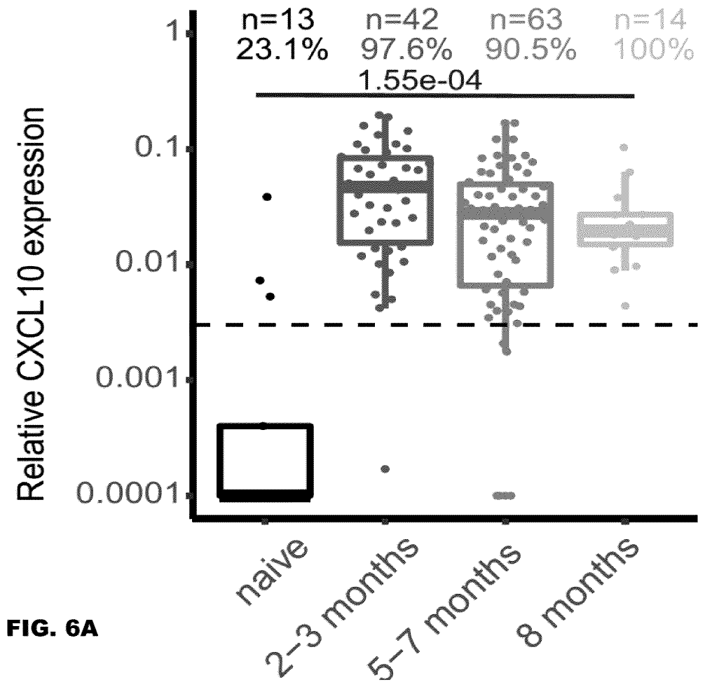

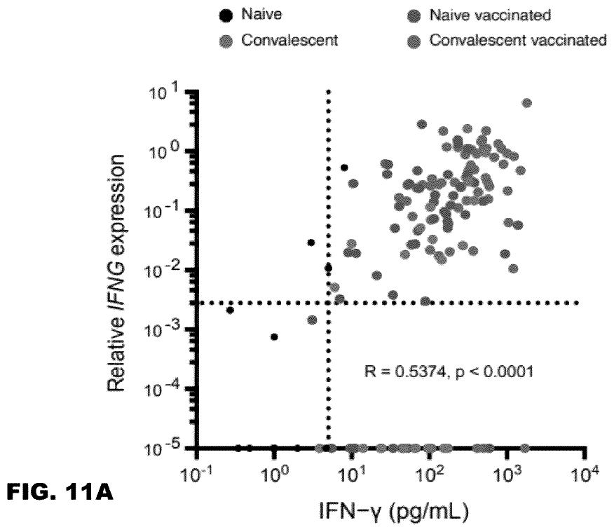

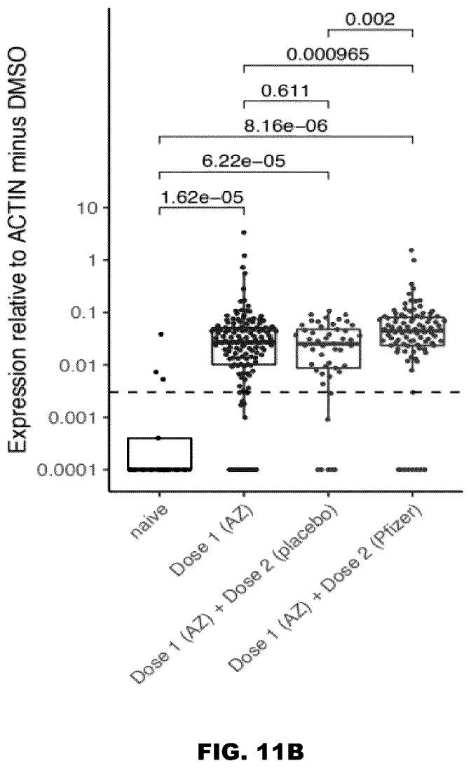

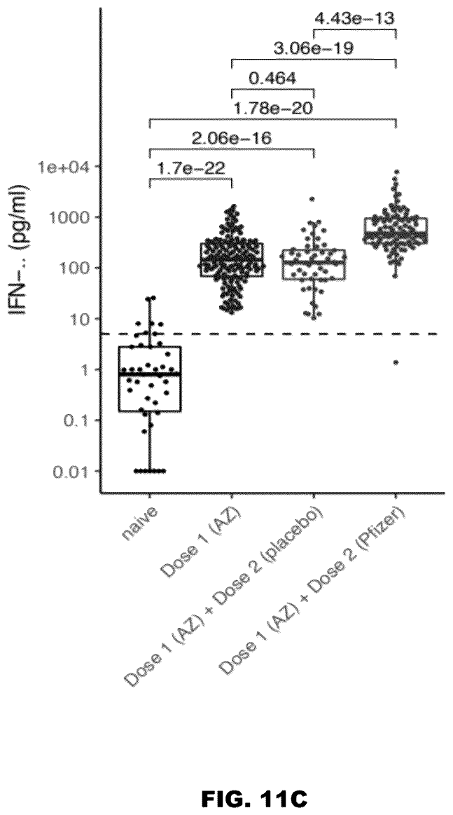

Finally, the inventors used the scalable qTACT and dqTACT tests, in parallel with ELLA and ELISpot, across multiple cohorts of subjects. First, the inventors used a cohort of 91 subjects (45 naïve and 46 COVID-19 convalescent), for which the inventors recently published the corresponding ELLA results (Lozano-Ojalvo, D. et al. (2021)). For this validation cohort, the inventors chose to quantify IFNG mRNA, rather than CXCL10, to include a gene that is expressed by antigen specific T cells and to directly correlate mRNA expression (qTACT) with IFN-γ protein secretion (ELLA). The cohort was recruited prior to vaccination and followed at day 10 and 20 after the first and second dose of the BNT162b2 vaccine and the data on IFN-γ an IL-2 cytokine secretion have been described separately (Lozano-Ojalvo, D. et al. (2021)). Consistent with the inventors previously published data, compared to naïve subjects, COVID-19 recovered individuals had a higher median expression of CXCL10 prior to vaccination (FIG. 5A). A similar trend was observed for IFNG levels, but the difference was not statistically significant (FIG. 5B; FIG. 11A). Importantly, quantification of the spike-specific T cell response by both CXCL10 and IFNG qPCR (qTACT) 10 and 20 days after the first and second dose, confirmed similar findings obtained by more traditional ELLA (FIG. 5C) and ELISpot (FIG. 5D) assays, with the clear advantage of cost effectiveness and scalability. The technical advantage of the present invention over ELLA or ELISpot is the ease of use of qPCR and, importantly, the internal normalization standard (i.e. ACTIN levels), which is absent in other more laborious methods of quantifying cellular immunity.

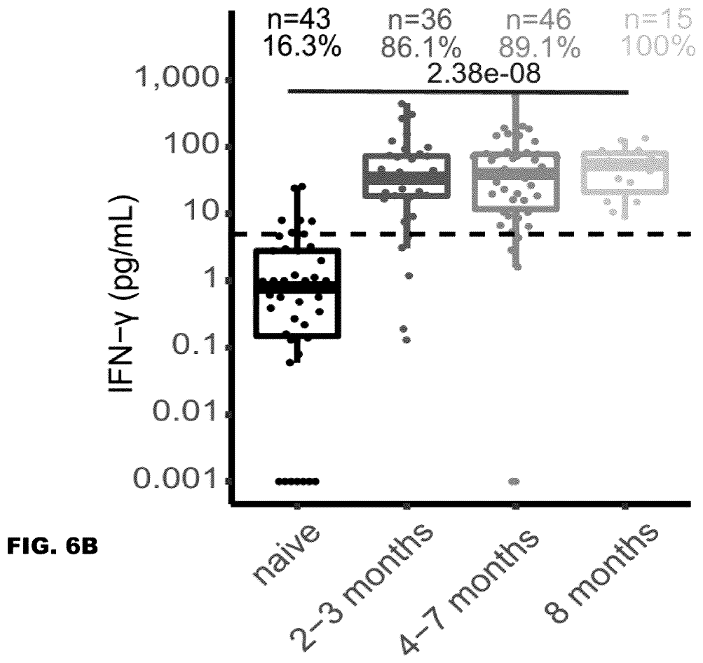

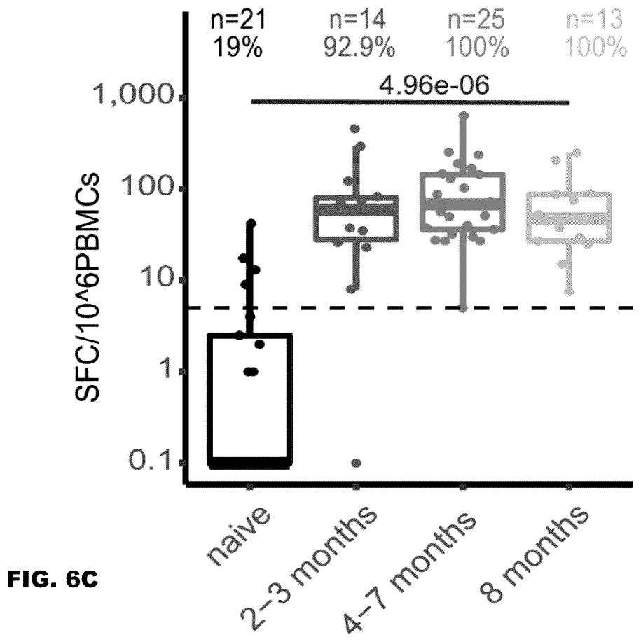

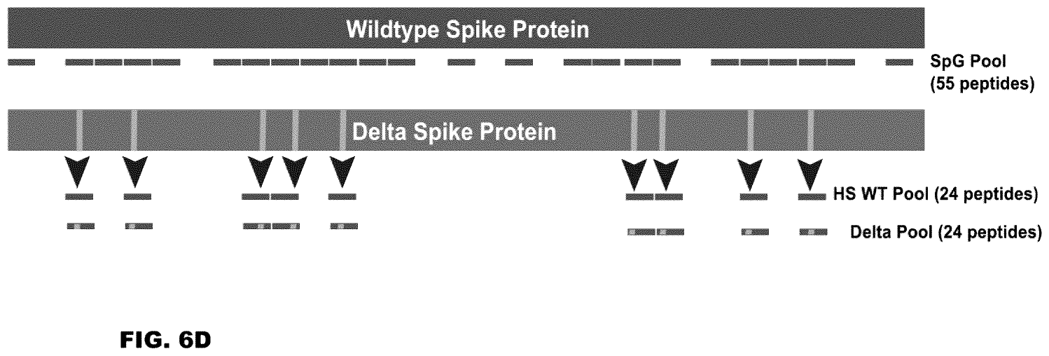

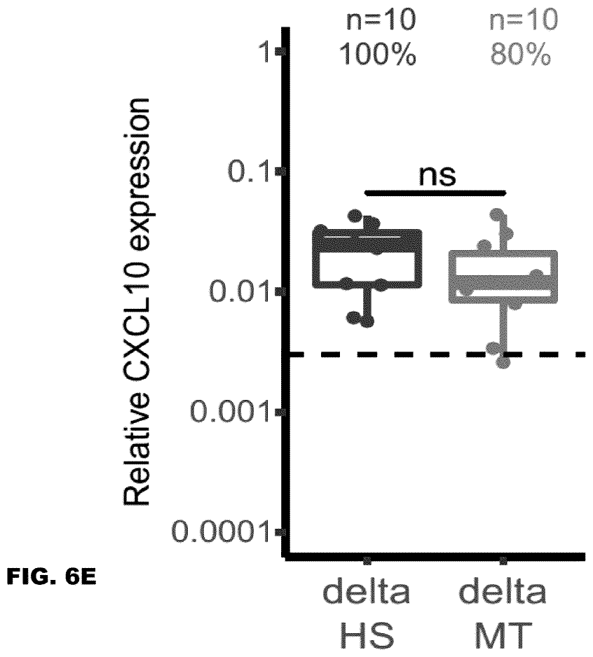

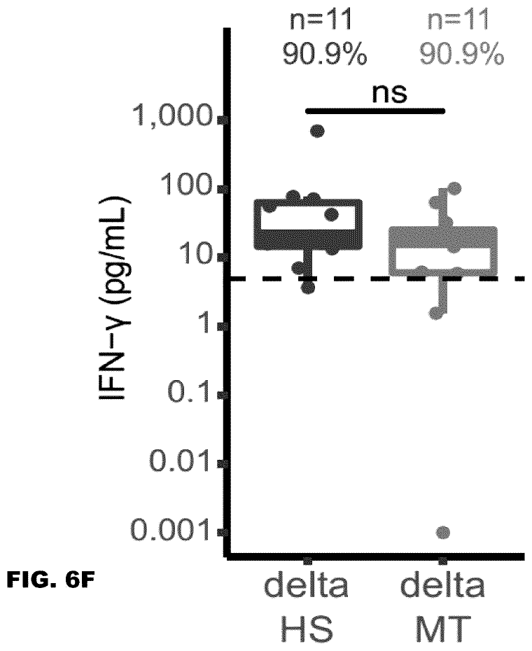

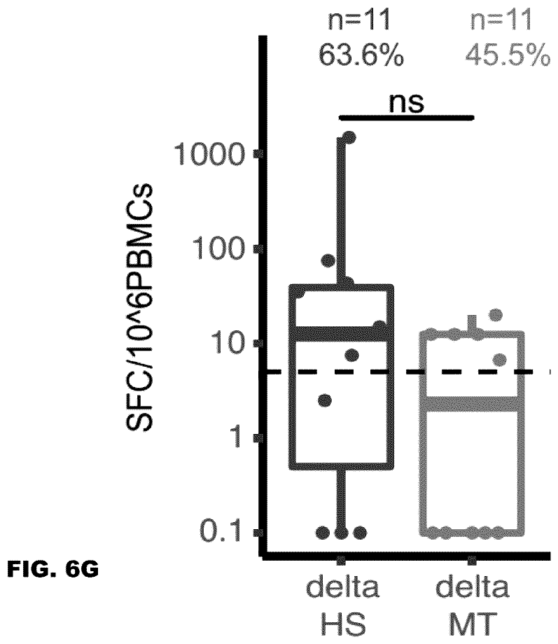

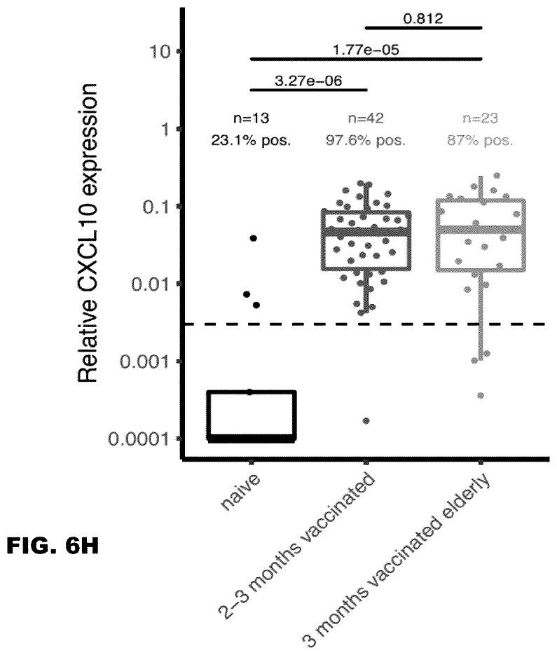

The inventors next used the dqTACT assay to quantify the levels of CXCL10, as a proxy for cellular immunity in vaccinated subjects at different time points after the second dose. The inventors observed a consistent detection of T cells above threshold by dqTACT (FIG. 6A; Table 6), ELLA (FIG. 6B; Table 7) and ELISpot (FIG. 6C; Table 8), up to 8 months post vaccination.

TABLE 6

| | |
|---|---|
| Average age | 33.22 |
| Age range | 17-60 |
| Percentage female (%) | 53.6 |
| Naïve N | 13 |
| 2-3 months vaccinated N | 42 |
| 5-7 months vaccinated N | 63 |
| 8 months vaccinated N | 14 |
| Total N | 132 |

| | Average CXCL10 | Standard |
|---|---|---|
| | expression | deviation |
| naive | 0.004 | 0.011 |

| | | | |
|---|---|---|---|
| 2-3 | months | 0.058 | 0.051 |
| 5-7 | months | 0.037 | 0.038 |
| 8 | months | 0.028 | 0.026 |

TABLE 7

| | |
|---|---|
| Average age | 34.88 |
| Age range | 17-65 |
| Percentage female (%) | 57.5 |
| Naïve N | 43 |
| 2-3 months vaccinated N | 36 |
| 4-7 monhts vaccinated N | 46 |
| 8 months vaccinated N | 15 |
| Total N | 140 |

| | Average IFN- γ | Standard |
|---|---|---|
| | (pg/mL) | deviation |
| naive | 2.7 | 5.43 |

| | | | |
|---|---|---|---|
| 2-3 | months | 65.95 | 92.89 |
| 4-7 | months | 66.59 | 93.97 |
| 8 | months | 55.98 | 40.09 |

TABLE 8

| | |
|---|---|
| Average age | 32.33 |
| Age range | 17-60 |
| Percentage female (%) | 53.8 |
| Naïve N | 21 |
| 2-3 months vaccinated N | 14 |
| 4-7 monhts vaccinated N | 25 |
| 8 months vaccinated N | 13 |
| Total N | 73 |

| | Average SFC/10\hat{ }6 | Standard |
|---|---|---|
| | PBMCs | deviation |
| naive | 4.44 | 9.84 |
| 2-3 months vaccinated | 96.44 | 126.76 |
| 4-7 months vaccinated | 109.06 | 129.15 |
| 8 months vaccinated | 73.17 | 74.27 |

### Example 5

### Assessment of Cellular Immunity

Finally, the inventors used the dqTACT assay to assess cellular immunity in: (i) a population of elderly individuals at 3 months post second dose. The inventors did not observe any appreciable difference compared to a younger population, and results were consistent with data obtained by ELLA and ELISpot (FIG. 6H-I); (ii) a larger cohort from a randomized, adaptive, phase 2 trial (CombiVacS) (Borobia, A. (2021) (FIG. 11B-C). The trial was designed to determine the reactogenicity and immunogenicity of a second dose of BNT162b2 (Comirnaty, BioNTech) in individuals who had received a first dose of ChAdOx1s (Vaxzevria, Astra Zeneca). The dqTACT assay reliably detected CXCL10 expression in 141 vaccinated individuals enrolled in this study. In line with previously published data from the CombiVacS trial where upregulation of IFN-γ was observed after heterologous vaccination, the inventors also observed a significant increase in CXCL10 expression in individuals who received a second dose (of BNT162b2) compared to those that received the placebo (only a first dose of ChAdOx1s, but no second dose) (FIG. 11B-C); (iii) a small population of vaccinated subjects stimulated with either a small subset of peptides covering the hotspots in the wildtype (Wuhan) or the delta variant (FIG. 6D; Table 9). The latter test was performed to assess whether the dqTACT assay could be rapidly deployed to assess the protection of individual T cell repertoires to emerging variants. The data demonstrates a slight, but not statistically significant decrease of T cell activation towards delta in subjects vaccinated with the wildtype (HS pool) strain consistently across the different platforms dqTACT (FIG. 6E), ELLA (FIG. 6F) and ELISpot (FIG. 6G). This is consistent with the substantial preservation of Spike-specific T cells response against mutated Spike protein present in different variants of concern (VOC) observed in most vaccinated and convalescent individuals.

TABLE 9

| Name | Average value | Standard devation | Assay |
|---|---|---|---|
| delta HS | 93.10157 | 202.6877058 | ELLA |
| delta MT | 25.76126 | 30.96419925 | ELLA |
| delta HS | 153.71182 | 447.1317576 | ELISpot |
| delta MT | 6.05 | 7.18542274 | ELISpot |
| delta HS | 0.02236 | 0.01310201 | dqTACT |
| delta MT | 0.01603 | 0.01300009 | dqTACT |

### Example 6

dqTACT Assay

The inventors have invented a rapid, user-friendly, accessible, scalable, and accurate diagnostic method for quantifying the amount of mRNA transcripts present in a tissue sample using RT PCR that can be performed using tissues such as whole blood or lymph without cellular lysis or pretreatment with proteases. The inventors hypothesized that proteins present in certain tissues, such as blood, inhibit PCR and prevent direct nucleotide amplification from unadulterated tissue. They sought to develop a more efficient RT PCR method that would be accurate and could be performed quickly using a variety of PCR platforms. The inventors tested an array of different conditions and additives to determine whether it was possible to perform RT-PCR directly on whole blood. The testing conditions included: pre-treatment of the tissue with heparinase; adding PCR enhancer cocktail; adding RNA/DNA shield (Zymo Research); pre-treating the blood with proteinase K; pre-treating blood with detergent; adding DMSO to the blood; and including CHELEX with detergent. The inventors surprisingly discovered that an effective way to detect mRNA transcripts (for ACTIN and CXCL10) in whole blood was to dilute a blood sample with a solution that can include detergent but does not require proteases. They discovered that diluting the tissue sample from between a ratio of 1:1 and 1:4 with water or water plus detergent but without proteases allowed the amplification of mRNA directly from the tissue. Below are some examples of formulations that were effective for amplifying mRNA directly from blood (either fresh or frozen).

Tissue Dilution Solution 1: Blood diluted 1:10 or 1:5 with water. Tissue Dilution Solution 2: Blood diluted 1:10 or 1:5 with a 1% or 2% solution of Tween20. Tissue Dilution Solution 3: Blood diluted 1:10 or 1:5 with a 1% solution of Triton. (Total solution volume for each is 100 ul.)

qPCR Mixtures: 2 ul of Tissue Dilution Solution is added per reaction container for a final volume of 20 ul. The remaining ingredients are:
10 ul SCRIPT Direct RT-qPCR ProbesMaster mix (Jena Bioscience)
0.1 ul ACTIN primer 1 (500 uM stock)
0.1 ul ACTIN primer 2 (500 uM stock)
0.1 ul CXCL10 primer 1 (100 uM stock)
0.1 ul CXCL10 primer 2 (100 uM stock)
0.05 ul ACTIN HEX probe (100 uM stock)
0.05 ul CXCL10 FAM probe (100 uM stock)
7.5 ul PEC-1 (Klentaq)

### Example 7

### SARS-CoV-2 Peptide Pools

Spike (1276 amino acids long), requires a total of 253 15-mer peptides overlapping by 10 amino acids to cover the entire protein. The inventors refined the pool (spike gold-SpG) encompassing 40.5% of the spike protein (55 peptides) (Le Bert, N. et al. (2021)). Spike gold encompasses most of the SARS-CoV2 spike epitope published to date. Pool HS contains peptides covering the non-conserved Spike-Wuhan regions affected by mutations present in the delta variant (24 peptides). Pool delta MT contains peptides from Pool delta HS with the amino acid mutations present in the Spike protein of the delta variant. A similar strategy (15-mer peptides) was also used to cover nucleoprotein (NP2) as previously described (Kilpeläinen, A. et al. (2021); Le Bert, N. et al. (2020)).

Whole Blood Culture with SARS-CoV-2 Peptide Pools

320 μl of whole blood drawn on the same day were mixed with 80 ul RPMI and stimulated with pools of SARS-CoV-2 peptides (S or NP; 2 μg/ml) or DMSO control at 37° C. After 15-17 hours of stimulation, the supernatant (plasma) was collected and stored at −80° C. until quantification of cytokines.

### RNA-Seq

Whole blood was treated with ACK lysis buffer for 15 minutes at RT to remove red blood cells. Cells were resuspended in Trizol (300 μL) and RNA was extracted using Zymo's Direct-zol extraction kit as per the manufacturer's instructions. Sequencing libraries were prepared from the eluted RNA using the Ovation Ultralow V2 DNA-Seq Library Preparation Kit following the manufacturer's instruction (NuGEN) at the Mount Sinai Oncological Sciences Sequencing Facility. Sequencing was performed on an Illumina NextSeq 500 instrument to produce 75-bp single-end reads. Demultiplexed FASTQ files were subsequently returned for analysis.

### RNA-Seq Data Analysis

Transcript expression was quantified from RNA-Seq data using Salmon 1.2.1 43 against an index built from the Ensembl (Patro, R. et al. (2017)) GRCh38 v99 transcriptome model with default parameters. Pseudocounts were imported into an R 4.0.3 environment (Love, M. I. et al. (2014)) and summarized to the gene level using tximeta 1.8.2. Differential expression analysis was conducted separately for naïve and convalescent individuals using DESeq2 1.3.0 (Love, M. I. et al. 2014)) with default parameters. Only protein-coding genes with at least 10 total counts were included for each analysis, and donor information was included in the design. P-values were corrected using the Benjamini-Hochberg method. Significantly upregulated genes were defined as those with an adjusted p-value≤0.05 and log 2FoldChange>1. Gene set enrichment analysis was performed using gprofiler2 0.2.0 using default parameters (Raudvere, U. et al. (2019)).

qTACT Assay

Samples used for RNA extraction were diluted 1:1 with RNA/DNA shield (Zymo) and incubated at room temperature with proteinase K at a 1:100 dilution (20 mg/ml stock). Samples were then frozen at −80° C. until RNA extraction could be performed. Samples stored in RNA/DNA shield were thawed at room temperature prior to RNA extraction. Samples were vortexed and mixed with Trizol reagent (Life Technologies) at a 1:1 dilution. After vortexing, samples were processed using Zymo's Direct-zol 96 well extraction kit as per the manufacturer's instructions. Eluted RNA was diluted in TE buffer, aliquoted, and stored at −80° C. or used immediately for qPCR analysis. Real-time quantification was performed on a BioRad CFX96/CFX384 or Hyris bCUBE 2.0. 5 ul of diluted RNA was used with the TaqPath 1-Step Multiplex MasterMix (Applied Biosystems) and primers/probes targeting ACTIN (internal control) and other target genes.

dqTACT Assay

Samples used for direct amplification from whole blood were diluted 1:4 and stored at −80° C. or used immediately for qPCR analysis. 2 ul of diluted whole blood was mixed with SCRIPT Direct RT-qPCR ProbesMaster (Jena Bioscience) and primers/probes targeting ACTIN (internal control) and other target genes, as described. Quantification was performed using the Hyris bCUBE 2.0, CFX96, CFX384 as specified.

### Cytokine Quantification and Analysis

Cytokine concentrations in the plasma were quantified using Ella with microfluidic multiplex cartridges measuring IFN-γ and IL-2 following the manufacturer's instructions (ProteinSimple, San Jose, California). The level of cytokines present in the plasma of DMSO controls was subtracted from the corresponding peptide pool stimulated samples.

### Spike-Specific IgG Quantification

The ACCESS SARS-CoV-2 CLIA (Beckman Coulter Inc., California, USA) was used for semiquantitative detection of IgG directed against S protein RBD using serum obtained from venipuncture blood. Samples were tested on a UniCel Dxl 800 high-performance analyser.

### Olink Immunoassay

Cytokine concentrations in the plasma were analyzed using Olink multiplex assay platform with Inflammatory panel (Olink Bioscience, Uppsala, Sweden), according to the manufacturer's instructions. The inflammatory panel includes 92 proteins associated with human inflammatory conditions. Briefly, an incubation master mix containing pairs of oligonucleotide-labeled antibodies to each protein was added to the samples and incubated for 16 hours at 4° C. Each protein was targeted with two different epitope-specific antibodies increasing the specificity of the assay. Presence of the target protein in the sample brought the partner probes in close proximity, allowing the formation of a double strand oligonucleotide polymerase chain reaction (PCR) target.

On the following day, the extension master mix in the sample initiated the specific target sequences to be detected and generated amplicons using PCR in 96 well plate. For the detection of the specific protein, Dynamic array integrated fluidic Circuit (IFC) 48×48 chip was primed, loaded with 45 protein specific primers and mixed with sample amplicons including three inter-plate controls (IPS) and three negative controls (NC). Real time microfluidic qPCR was performed in Biomark (Fluidigm, San Francisco, CA) for the target protein quantification. Data were analyzed using Real time PCR analysis software via ΔΔCt method and Normalized Protein Expression (NPX) manager. Data were normalized using internal controls in every single sample, inter-plate control (IPC) and negative controls and correction factor and expressed as Log 2 scale which is proportional to the protein concentration. One NPX difference equals to the doubling of the protein concentration.

### ELISpot

Enzyme-linked immunospot (ELISpot) flat-bottomed, 96-well nitrocellulose plates (MAHA S4510; Millipore) were coated with IFN-γ mAb (2 μg/ml, 1-DIK; MABTECH, Stockholm) and incubated for 2 hours at 37° C. After washing with PBS, plates were blocked with 10% human AB serum for 2 hours at 37° C. Cells were washed, concentrated, plated into each well titrating down in two-fold serial dilutions starting from 400,000 PBMCs, in the presence of peptides, negative control (Dimethyl sulfoxide (DMSO) and positive control phorbol 12-myristate 13-acetate and ionomycin (PMA)/ionomycin). Plates were incubated for 24 hours in a CO2 incubator. Post incubation, the plates were semi-automatically washed with PBS, and then IFN-γ mAb (0.2 μg/ml, 7-B6-1-biotin; MABTECH) was added to each well. After incubation for 2 hours at 37° C., plates were washed and incubated with streptavidin-alkaline phosphatase (1 μg/ml; Roche) for 1 hour at room temperature. After washing unbound streptavidin-alkaline phosphatase, substrate (5-bromo-4-chloro-3-indolyl phosphate/NBT; Sigma-Aldrich) was added and incubated for 12-15 min for spots to develop. Post incubation, plates were washed to remove substrate and dark-violet spots were evaluated using the C.T.L. Immunospot analyzer and software (Cellular Technology Limited) as routinely performed in the lab (Rydyznski Moderbacher, C. et al. (2020)).

### Flow Cytometry

Flow cytometry analyses were carried out in whole blood samples labeled for viability (Blue Live/dead fixable dye, BioLegend, San Diego, CA), washed, and stained for extracellular markers for 45 min at 4° C. For intracellular staining, samples were fixed in 4% paraformaldehyde (Electron Microscopy Services, Hatfield, PA), and treated with permeabilization buffer (eBioscience, San Diego, CA) before staining with labeled antibodies to detect intracellular cytokines, CD154, and CXCL10. Cells were subsequently acquired on a 5-lasers Cytek Aurora device (Cytek Biosciences, Fremont, CA) and data analyzed on Cytobank (https://cytobank.org/). For sorting experiments, whole blood samples were exclusively stained for the surface markers CD16, CD3, HLA-DR, CD66b, CD15, CD8, CD4, and CD14. Four different populations (neutrophils, monocytes, CD4+, and CD8+ T cells) were sorted with purities >96% by using a FACS Aria II Cell Sorter device (BD Biosciences, Franklin Lakes, NJ).

### PCR Primers

TABLE 10

| | | | SEQ ID |
|---|---|---|---|
| Target | Type | Sequence | NO(s): |
| ACTIN | primer 1 | CCTTGCACATGCCGGAG | 1 |
| ACTIN | primer 2 | ACAGAGCCTCGCCTTTG | 2 |
| ACTIN | probe | TCATCCATGGTGAGCTGGCGG | 3 |
| CXCL10 | primer 1 | CCATTCTGATTTGCTGCCTTATC | 4 |
| CXCL10 | primer 2 | TACTAATGCTGATGCAGGTACAG | 5 |
| CXCL10 | probe | AGTGGCATTCAAGGAGTACCTCTCTCT | 6 |
| CXCL9 | primer 1 | AAT ACAGGAGTGACTTGGAACT | 7 |
| CXCL9 | primer 2 | AACAGCGACCCTTTCTCAC | 8 |
| CXCL9 | probe | TCTTGCTGG TTCTGATTGGAGTGCA | 9 |
| ANKRD22 | primer 1 | AGTTAATGCTACAGATTGTTATGGC | 10 |
| ANKRD22 | primer 2 | TCCGTGCAAT ATCCAGTGAG | 11 |
| ANKRD22 | probe | CCCGTGCAGACCCCACAAT AAAGA | 12 |
| GBP1 | primer 1 | GATCCAGTGAGAGACACAGAA | 13 |
| GBP1 | primer 2 | CATGTCCAGGCTGTTCCC | 14 |
| GBP1 | probe | TGCTAGAAGCCAGTGCTCGTGAAC | 15 |
| IFNG | primer 1 | TCGGTAACTGACTTGAATGTCCAACGC | 16 |
| IFNG | primer 2 | CGACAGTTCAGCCATCACTT | 17 |
| IFNG | probe | TCGGTAACTGACTTGAATGTCCAACGC | 16 |
| IL2 | primer 1 | AAACTCACCAGGATGCTCAC | 18 |
| IL2 | primer 2 | CTCCAGAGGTTTGAGTTCTTCT | 19 |
| IL2 | probe | TT ACA TGCCCAAGAAGGCCACAGA | 20 |

### Statistical Analyses

All illustrations were prepared using ggplot2 and ggpubr in an R 4.0.3. Statistical significance in figures is reported as follows: *p<=0.05, **p<=0.01, ***p<=0.001, *** p<=0.0001. For RNA-Seq data (FIG. 1B and FIG. 4), displayed p-values were generated using DESeq2 as described in the RNA-Seq Data Analysis section. For all plots, displayed p-values were generated using a two-sided Wilcoxon Rank Sum test in R and corrected using the Benjamini-Hochberg method. ROC curves and associated 95% CIs for each assay were computed in R using the pROC package (v1.18.0) (Robin, X. et al. (2011)). Fisher exact tests and associated odds ratios were computer in R using the fisher.test function using a two-sided approach.

1. A method for detecting in a subject the presence of T-cells specific for a particular virus, wherein: a tissue sample from said subject has been stimulated for a period of time via incubation with a pool of polypeptides encoded by sequences unique to said virus; at least one quantitative PCR (qPCR) assay has been performed on said stimulated tissue sample; results have been obtained that quantify the relative concentration of CXCL10 mRNAs in the sample; said results have been compared with a reference standard; and the results of the comparison indicate whether said subject has T-cells specific for said virus.

2. The method of claim 1, wherein said virus is SARS-CoV-2 and wherein said pool comprises polypeptides derived from at least one SARS-CoV-2 viral spike protein, from at least one SARS-CoV-2 nucleoprotein, at least one SARS-CoV-2 non-structural protein, or combinations thereof.

3. The method of claim 2, wherein the results of said comparison with said reference standard indicate whether said subject has T-cells that are specific for mutated variants of SARS-CoV-2.

4. The method of claim 2, wherein said tissue sample is pretreated by mixing said tissue sample with a buffer to generate a reaction mixture, wherein said buffer optionally comprises a final detergent concentration of about 0.0 g/mL to about 0.1 g/mL.

5. The method of claim 2, wherein said tissue sample is pretreated by mixing said tissue sample with a buffer to generate a reaction mixture, wherein said buffer comprises the detergent Tween-20 or Triton, and wherein said reaction mixture comprises a final detergent concentration of about 0.0 g/mL to about 0.1 g/mL.

6. The method of claim 2, wherein said tissue sample is pretreated by mixing said tissue sample with a buffer to generate a reaction mixture, wherein said buffer comprises Tween-20, and wherein said pretreatment mixture comprises a final detergent concentration of about of about 0.0 g/mL to about 0.1 g/mL.

7. The method of claim 2, wherein prior to performing qPCR the blood is diluted between a ratio of about 1:1 to about 1:5 to make the sample mixture.

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. A method for detecting an immune response to SARS-CoV-2 in a subject, wherein: a tissue sample from the subject has been stimulated via incubation with a pool of polypeptides derived from SARS-CoV-2 or a mutated variant thereof for a period of time; at least one quantitative PCR (qPCR) assay has been performed on said stimulated tissue sample; results have been obtained that quantify the relative concentration of CXCL10 mRNAs in the sample; said results have been compared with a reference standard; the results of the comparison indicate whether to treat the subject for SARS-CoV-2.

22. The method of claim 21, wherein said pool of SARS-CoV-2 derived polypeptides contains fragments or complete polypeptides from at least one spike protein, at least one nucleoprotein, at least one non-structural protein, or combinations thereof.

23. The method of claim 22 further comprising providing a SARS-CoV-2 vaccination to said subject.

24. (canceled)

25. (canceled)

26. A method for treating SARS-CoV-2 infection in a subject, wherein: a tissue sample from said subject has been stimulated via incubation with a pool of polypeptides derived from SARS-CoV-2 or a mutated variant thereof for a period of time; at least one quantitative PCR (qPCR) assay has been performed on said stimulated tissue sample; results have been obtained that quantify the relative concentration of CXCL10 mRNAs in the sample; said results have been compared with a reference standard; said results of the comparison indicate whether to treat the subject for a SARS-CoV-2 infection; and providing or withholding treatment for SARS-CoV-2.

27. The method of claim 26, wherein said pool of polypeptides contains fragments or complete polypeptides from at least one spike protein, at least one nucleoprotein, at least one non-structural protein, or combinations thereof.

28. The method of claim 27, comprising providing a SARS-CoV-2 vaccination to said subject.

29. The method of claim 27, comprising administering to said subject a therapeutic selected from the group consisting of: antivirals, corticosteroids, monoclonal antibodies, NSAIDs, convalescent plasma, or antidepressants.

30. The method of claim 27, wherein said tissue sample is blood and said blood has been diluted prior to the performance of the at least one qPCR assay.

31. A method for gauging the immune status of a subject with respect to a specific virus, wherein: a tissue sample from said subject has been stimulated via incubation with a pool of polypeptides derived from sequences specific to said virus or mutated variants of said virus for a period of time; at least one quantitative PCR (qPCR) assay has been performed on said stimulated tissue sample; results have been obtained that quantify the concentration of relative CXCL10 mRNAs in the sample; said results have been compared with a reference standard; said results of the comparison indicate whether to treat the subject for an infection by said virus.

32. The method of claim 31, wherein said virus is SARS-CoV-2 and said pool of peptides contains fragments or complete polypeptides derived from at least one spike protein, at least one nucleoprotein, or combinations thereof.
